(19) United States
(12) Patent Application Publication
(10) Pub. No.: US 2025/0032735 A1
(43) Pub. Date: Jan. 30, 2025

(54) SYSTEMS AND METHODS FOR DETERMINING AND PROVIDING AN INDICATION OF WELLBEING OF A USER

(71) Applicant: ResMed Digital Health Inc., San Diego, CA (US)

(72) Inventors: Liam John Keast, Halifax (CA); Keegan Duane Dsouza, Pompano Beach, FL (US); Dylan Hermes da Fonseca Beadle, San Diego, CA (US); Ray Collins, San Diego, CA (US)

(21) Appl. No.: 18/783,748

(22) Filed: Jul. 25, 2024

Related U.S. Application Data
(60) Provisional application No. 63/516,361, filed on Jul. 28, 2023.

(51) Int. Cl. A61M 16/00; A61M 16/06; A61M 16/10; G16H 10/60; G16H 20/40

(52) U.S. Cl. CPC A61M 16/0066; A61M 16/0003; A61M 16/024; A61M 16/0622; A61M 16/109; G16H 10/60; G16H 20/40; A61M 2016/0027; A61M 2016/0033; A61M 2202/0007; A61M 2202/02; A61M 2205/103; A61M 2205/3303; A61M 2205/3306; A61M 2205/3327; A61M 2205/3334; A61M 2205/3368; A61M 2205/3375; A61M 2205/36; A61M 2205/505; A61M 2230/04; A61M 2230/10; A61M 2230/20; A61M 2230/50; A61M 2230/63

(57) ABSTRACT

Systems and methods involve determining one or more first measures of wellbeing of a user with sleep disordered breathing during an off period when the user is not adhering to a positive airway pressure therapy. The systems and methods also involve determining one or more second measures of the wellbeing of the user during an on period when the user is at least partially adhering to the positive airway pressure therapy. The systems and methods also involve presenting an indication to the user on how at least partially adhering to the positive airway pressure therapy improves the wellbeing of the user by comparing the one or more first measures to the one or more second measures.

### PRIORITY

This application claims priority from and benefit of U.S. Provisional Patent Application Ser. No. 63/516,361, filed Jul. 28, 2023. The contents of that application are hereby incorporated by reference in their entirety.

### TECHNICAL FIELD

The present disclosure relates generally to systems and methods for measuring wellbeing of a user, and more particularly, to systems and methods for showing how adhering to positive airway pressure therapy improves wellbeing.

### BACKGROUND

Many individuals suffer from sleep-related and/or respiratory-related disorders such as, for example, Sleep Disordered Breathing (SDB), which can include Obstructive Sleep Apnea (OSA), Central Sleep Apnea (CSA), other types of apneas, such as mixed apneas and hypopneas, Respiratory Effort Related Arousal (RERA), and snoring. In some cases, these disorders manifest, or manifest more pronouncedly, when the individual is in a particular lying/sleeping position. These individuals may also suffer from other health conditions (which may be referred to as comorbidities), such as insomnia (e.g., difficulty initiating sleep, frequent or prolonged awakenings after initially falling asleep, and/or an early awakening with an inability to return to sleep), Periodic Limb Movement Disorder (PLMD), Restless Leg Syndrome (RLS), Cheyne-Stokes Respiration (CSR), respiratory insufficiency, Obesity Hyperventilation Syndrome (OHS), Chronic Obstructive Pulmonary Disease (COPD), Neuromuscular Disease (NMD), rapid eye movement (REM) behavior disorder (also referred to as RBD), dream enactment behavior (DEB), hypertension, diabetes, stroke, and chest wall disorders.

These disorders are often treated using a respiratory therapy system (e.g., a continuous positive airway pressure (CPAP) system), which delivers pressurized air to aid in preventing the individual's airway from narrowing or collapsing during sleep. However, some users find such systems to be uncomfortable, difficult to use, expensive, aesthetically unappealing, and/or fail to perceive the benefits associated with using the system. As a result, some users will elect not to use the respiratory therapy system or discontinue use of the respiratory therapy system absent a demonstration of the severity of their symptoms when respiratory therapy treatment is not used or encouragement or affirmation that the respiratory therapy system is improving their sleep quality and reducing the symptoms of these disorders. The present disclosure is directed to solving these and other problems.

### SUMMARY

According to some implementations of the present disclosure, a method includes determining one or more first measures of wellbeing of a user with sleep disordered breathing during an off period when the user is not adhering to a respiratory pressure therapy. The method also includes determining one or more second measures of the wellbeing of the user during an on period when the user is at least partially adhering to the respiratory pressure therapy. The method also includes presenting an indication to the user on how at least partially adhering to the respiratory pressure therapy improves the wellbeing of the user by comparing the one or more first measures to the one or more second measures.

According to some aspects of the implementations, the one or more first measures of wellbeing relate to sleep quality and are based on information obtained from one or more devices other than a sleep respiratory device providing the respiratory pressure therapy. The one or more second measures of the wellbeing relate to sleep quality and are based on information obtained from sleep respiratory device providing the respiratory pressure therapy. According to some aspects of the implementations, each type of first measure of the one or more first measures can be normalized among its respective type of the first measure to compare the respective type of the first measure with the one or more first measures. Further, each type of second measure of the one or more second measures can be normalized among its respective type of the second measure to compare the respective type of the second measure with the one or more second measures. Even further, each type of first measure of the one or more first measures can be normalized among its respective type of the first measure to compare the respective type of the first measure with the one or more second measures, and each type of second measure of the one or more second measures can be normalized among its respective type of the second measure to compare the respective type of the second measure with the one or more first measures. According to some aspects of the implementations, a format of the indication is based on a health history of the user, one or more current health issues of the user, a type of sleep disordered breathing, or a combination thereof. According to some aspects of the implementations, the indication is presented on a respiratory therapy device the user uses for the respiratory pressure therapy. According to some aspects of the implementations, the indication is presented across multiple devices associated with the user. According to some aspects of the implementations, a format of the indication varies depending on a device type for the multiple devices associated with the user. According to some aspects of the implementations, the method includes collecting information from one or more devices associated with the user separate from a respiratory therapy device that provides the positive airway pressure to the user. The information can be collected at least during the off period. Alternatively, the information can be collected during the off period and the on period. According to some aspects of the implementations, the one or more devices are one or more health trackers, one or more smart devices, one or more home assistants, one or more exercise equipment, or a combination thereof. According to some aspects of the implementations, the one or more devices belong to one or more health ecosystems. According to some aspects of the implementations, the one or more first measures, the one or more second measures, or a combination thereof are determined based on one or more common objective measures of the wellbeing of the user. According to some aspects of the implementations, the one or more common objective measures include measures of consistency and/or punctuality in one or more daily routines, results of one or more cognitive tests, performance during one or more games, performance during one or more predefined routines, or a combination thereof. According to some aspects of the implementations, the one or more daily routines include a daily routine of waking up and getting ready for work, including time to get out of bed, time to take a shower, and time to brush teeth. According to some aspects of the implementations, the one or more cognitive tests include reaction time tests, reading time tests, and reading comprehension tests. According to some aspects of the implementations, the one or more first measures, the one or more second measures, or a combination thereof are determined based on one or more subjective measures provided by the user. According to some aspects of the implementations, the one or more first measures, the one or more second measures, or a combination thereof are determined based on health information associated with the user and collected by one or more smart devices. According to some aspects of the implementations, the one or more second measures are determined based on health information associated with the user and collected by a respiratory therapy system used by the user for at least partially adhering to the respiratory pressure therapy. According to some aspects of the implementations, the one or more first measures of wellbeing and the one or more second measures of wellbeing relate to fatigue of the user. According to some aspects of the implementations, the method also includes determining one or more contributing factors that affect the wellbeing of the user during the off period, the on period, or a combination thereof. The indication to the user correlates the one or more contributing factors with the one or more first measures of wellbeing, the one or more second measures of wellbeing, or a combination thereof. According to some aspects of the implementations, the one or more contributing factors include one or more environmental factors.

According to some implementations of the present disclosure, a system includes a memory and a control system. The memory stores machine-readable instructions. The control system includes one or more processors configured to execute the machine-readable instructions to determine one or more first measures of wellbeing of a user with sleep disordered breathing during an off period when the user is not adhering to a respiratory pressure therapy. The one or more processors are further configured to execute the machine-readable instructions to determine one or more second measures of the wellbeing of the user during an on period when the user is at least partially adhering to the respiratory pressure therapy. The one or more processors are further configured to execute the machine-readable instructions to present an indication to the user on how at least partially adhering to the respiratory pressure therapy improves the wellbeing of the user by comparing the one or more first measures to the one or more second measures.

The above summary is not intended to represent each implementation or every aspect of the present disclosure. Additional features and benefits of the present disclosure are apparent from the detailed description and figures set forth below.

### BRIEF DESCRIPTION OF THE DRAWINGS

FIG. 1 is a functional block diagram of a system, according to some implementations of the present disclosure;

FIG. 2 is a perspective view of at least a portion of the system of FIG. 1, a user, and a bed partner, according to some implementations of the present disclosure;

FIG. 3A is a perspective view of a respiratory therapy device of the system of FIG. 1, according to some implementations of the present disclosure;

FIG. 3B is a perspective view of the respiratory therapy device of FIG. 3A illustrating an interior of a housing, according to some implementations of the present disclosure;

FIG. 4A is a perspective view of a user interface, according to some implementations of the present disclosure;

FIG. 4B is an exploded view of the user interface of FIG. 4A, according to some implementations of the present disclosure;

FIG. 5 illustrates an exemplary timeline for a sleep session, according to some implementations of the present disclosure;

FIG. 6 illustrates an exemplary hypnogram associated with the sleep session of FIG. 5, according to some implementations of the present disclosure; and

FIG. 7 is a process flow diagram for a method for presenting an indication to the user on how at least partially adhering to the respiratory pressure therapy improves the wellbeing of the user according to some implementations of the present disclosure.

While the present disclosure is susceptible to various modifications and alternative forms, specific implementations and embodiments thereof have been shown by way of example in the drawings and will herein be described in detail. It should be understood, however, that it is not intended to limit the present disclosure to the particular forms disclosed, but on the contrary, the present disclosure is to cover all modifications, equivalents, and alternatives falling within the spirit and scope of the present disclosure as defined by the appended claims.

### DETAILED DESCRIPTION

Many individuals suffer from sleep-related and/or respiratory disorders, such as Sleep Disordered Breathing (SDB) such as Obstructive Sleep Apnea (OSA), Central Sleep Apnea (CSA) and other types of apneas, Respiratory Effort Related Arousal (RERA), snoring, Cheyne-Stokes Respiration (CSR), respiratory insufficiency, Obesity Hyperventilation Syndrome (OHS), Chronic Obstructive Pulmonary Disease (COPD), Periodic Limb Movement Disorder (PLMD), Restless Leg Syndrome (RLS), Neuromuscular Disease (NMD), and chest wall disorders.

Obstructive Sleep Apnea (OSA), a form of Sleep Disordered Breathing (SDB), is characterized by events including occlusion or obstruction of the upper air passage during sleep resulting from a combination of an abnormally small upper airway and the normal loss of muscle tone in the region of the tongue, soft palate and posterior oropharyngeal wall. More generally, an apnea generally refers to the cessation of breathing caused by blockage of the air (Obstructive Sleep Apnea) or the stopping of the breathing function (often referred to as Central Sleep Apnea). CSA results when the brain temporarily stops sending signals to the muscles that control breathing. Typically, the individual will stop breathing for between about 15 seconds and about 30 seconds during an obstructive sleep apnea event.

Other types of apneas include hypopnea, hyperpnea, and hypercapnia. Hypopnea is generally characterized by slow or shallow breathing caused by a narrowed airway, as opposed to a blocked airway. Hyperpnea is generally characterized by an increase depth and/or rate of breathing. Hypercapnia is generally characterized by elevated or excessive carbon dioxide in the bloodstream, typically caused by inadequate respiration.

A Respiratory Effort Related Arousal (RERA) event is typically characterized by an increased respiratory effort for ten seconds or longer leading to arousal from sleep and which does not fulfill the criteria for an apnea or hypopnea event. RERAs are defined as a sequence of breaths characterized by increasing respiratory effort leading to an arousal from sleep, but which does not meet criteria for an apnea or hypopnea. These events fulfil the following criteria: (1) a pattern of progressively more negative esophageal pressure, terminated by a sudden change in pressure to a less negative level and an arousal, and (2) the event lasts ten seconds or longer. In some implementations, a Nasal Cannula/Pressure Transducer System is adequate and reliable in the detection of RERAs. A RERA detector may be based on a real flow signal derived from a respiratory therapy device. For example, a flow limitation measure may be determined based on a flow signal. A measure of arousal may then be derived as a function of the flow limitation measure and a measure of sudden increase in ventilation. One such method is described in WO 2008/138040 and U.S. Pat. No. 9,358,353, assigned to ResMed Ltd., the disclosure of each of which is hereby incorporated by reference herein in their entireties.

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterized by repetitive de-oxygenation and re-oxygenation of the arterial blood.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common, such as increased resistance to air movement, extended expiratory phase of respiration, and loss of the normal elasticity of the lung. COPD encompasses a group of lower airway diseases that have certain characteristics in common, such as increased resistance to air movement, extended expiratory phase of respiration, and loss of the normal elasticity of the lung.

Neuromuscular Disease (NMD) encompasses many diseases and ailments that impair the functioning of the muscles either directly via intrinsic muscle pathology, or indirectly via nerve pathology. Chest wall disorders are a group of thoracic deformities that result in inefficient coupling between the respiratory muscles and the thoracic cage.

These and other disorders are characterized by particular events (e.g., snoring, an apnea, a hypopnea, a restless leg, a sleeping disorder, choking, an increased heart rate, labored breathing, an asthma attack, an epileptic episode, a seizure, or any combination thereof) that occur when the individual is sleeping.

The Apnea-Hypopnea Index (AHI) is an index used to indicate the severity of sleep apnea during a sleep session. The AHI is calculated by dividing the number of apnea and/or hypopnea events experienced by the user during the sleep session by the total number of hours of sleep in the sleep session. The event can be, for example, a pause in breathing that lasts for at least 10 seconds. An AHI that is less than 5 is considered normal. An AHI that is greater than or equal to 5, but less than 15 is considered indicative of mild sleep apnea. An AHI that is greater than or equal to 15, but less than 30 is considered indicative of moderate sleep apnea. An AHI that is greater than or equal to 30 is considered indicative of severe sleep apnea. In children, an AHI that is greater than 1 is considered abnormal. Sleep apnea can be considered “controlled” when the AHI is normal, or when the AHI is normal or mild. The AHI can also be used in combination with oxygen desaturation levels to indicate the severity of Obstructive Sleep Apnea.

In consideration of the foregoing disorders, the present disclosure involves systems and methods for showing how adhering to positive airway pressure therapy improves wellbeing despite a user suffering from one or more of the foregoing disorders. The systems and methods of the present disclosure collect information associated with a user that relate to the wellbeing of the user. The information collected can be from periods of time during an off period, when the user is not adhering to a positive airway pressure therapy. The information collected can also be from periods of time during an on period, when the user is at least partially or fully adhering to the positive airway pressure therapy. Thereafter, an indication can be presented to the user to show how at least partially (or fully) adhering to the positive airway pressure therapy improves or imporved the wellbeing of the user. The indication compares the one or more first measures to the one or more second measures. During the off period(s), the information can be collected from various sensors associated with and/or near the user, which have the ability to collect information that relates directly or indirectly to the wellbeing of the user, as discussed further below. During the on period(s), the information can be collected from a respiratory therapy system that provides the positive airway pressure therapy, in addition to or without the information collected from the various sensors associated with and/or near the user used during the off period(s).

The information collected when the user is not adhering to a respiratory pressure therapy can be collected from various sources, such as, but not limited to, information from one or more smart devices; one or more smart phones (e.g., iPhone, etc.); one or more health trackers (e.g., Apple Watch, Fitbit, etc.); one or more pieces of exercise equipment; and/or one or more health ecosystems (e.g., Apple Health, Google Fit, etc.). According to some aspects, the information can be collected from systems and/or devices that are for another purpose different than determining wellbeing but otherwise can provide information on the same. For example, certain cars being sold and/or developed currently track how sleepy a user is while driving a vehicle. Such information can be collected for the purpose of providing information according to aspects of the present disclosure regarding the wellbeing of the user, where the wellbeing is tied specifically to fatigue or energy levels.

Various activities that a user performs throughout the day can reveal information on wellbeing, such as how well the user exercises during the day, how well the user performs certain activities during the day, the user's ability to concentrate during the day or during specific periods of time (e.g., work), the user's ability to do household chores, the user's ability to perform set tasks or goals throughout the day, etc. Any information that can be tracked and compared over time, during periods on and off positive airway pressure therapy, and that relate to the wellbeing of the user can be used, such as keystroke data at work or at home, data in relation to how quickly something is read (e.g., a newspaper article, a standardized paragraph or page of text, etc.), and the like, as further discussed below.

The information collected when the user is adhering to a respiratory pressure therapy can be collected from various sources, such as, but not limited to, the same sources of information for when the user is not adhering to a respiratory pressure therapy. The information can be collected from other sources, such as, but not limited to, data from the device providing positive airway pressure (e.g., respiratory therapy system or respiratory therapy device). Such data can include baseline data from the device providing positive airway pressure, including simply a start date and continued use information. Specific components of a respiratory therapy system that are unrelated specifically to providing positive airway pressure, such as a mask or user interface that can track movement, stages of sleep, heart rate, etc., can also provide the data. According to some implementations, the data can come instead or in addition from an application that is associated with receiving positive airway pressure, such as my Air™ patient support software and application provided by ResMed, which can collect holistic health information.

By collecting information on a user's wellbeing when the user is both adhering and not adhering to respiratory pressure therapy, the systems and methods of the present disclosure can correlate information when on and off therapy for showing relationships between health and positive airway pressure therapy. The relationships can be shown for the purpose of showing the benefit of respiratory pressure therapy, with the benefit being tied specifically to the wellbeing of the user. Such benefits, or the lack thereof, can be shown depending on the specific patient, taking into specific considerations, such as different health histories, current health issues, different types of sleep disordered breathing, etc. According to some aspects, the relationships can be presented to the user at different levels of granularity.

Information that relates to the wellbeing of a user can come in various different forms and be collected from various different devices and systems. To be able to compare information on wellbeing from the different systems and devices, the methods and devices of the present disclosure can normalize the collected information on wellbeing. Normalization can be performed based on averaging, based on crowd-sourced or personalized max/min information, and the like for the purpose of converting information that relates to wellbeing, such as activity or fatigue level, to a form that can be compared across modalities. For example, how quickly a user completes an online activity every morning can be compared to an average time or compared to a maximum and minimum time to normalize that information. Once normalized, the information on how long a user takes to complete the online activity can be compared to another activity or information, regardless of whether that activity or information relates to, for example, time. In a specific case, how well a user adheres to ideal sleep stages during sleep can indicate or relate to the user's wellbeing, such as fatigue level. However, the adherence to ideal sleep states during sleep can be difficult to compare to how quickly a user completes an online activity. Thus, normalizing the two metrics provides a way that standardizes each generally to wellbeing, so that they can be used together to provide an indication to a user that relates to the user's overall wellbeing, as defined by both metrics.

According to some implementations, the systems and methods of the present disclosure provide an activity metric that captures fatigue and/or energy level of the user. Through the activity metric, the user is shown how adhering and not adhering to positive airway pressure therapy affects his or her fatigue or energy level. The activity can be presented to the user through various interfaces, such as through a computing device's interface (e.g., computer, smartphone, etc.) and/or through one or more devices that can be specially configured to present the activity metric (e.g., mood or activity ring, an avatar, etc.). According to some implementations, the activity metric can be in the form of a forecasted trend line versus actual data, which can be broken down into how on-therapy sleep versus off-therapy sleep impacts data from a fitness activity.

The wellbeing of a user, as it relates to the present application, can primarily be the physical fatigue level of the user, such as how sleepy or energetic the user feels. However, the wellbeing of the user can relate to other health and/or physiological conditions of the user, such as an emotional fatigue level, how healthy the user feels, how well the user can concentrate, etc. According to some implementations, wellbeing can be physical health, including aspects such as nutrition, exercise, sleep, and access to healthcare; emotional health, including aspects such as stress management, emotional regulation, and the ability to form and maintain positive relationships; financial stability, including aspects such as financial security, which can reduce stress and provide a sense of stability, to name a few examples.

According to some implementations, a user can have set routines that can be used for determining the wellbeing. For example, a user may have a morning ritual or routine, which can be any repeatable process or one or more discrete aspects of the process that are repeatable. Aspects of the morning rituals can be measured and/or analyzed for determining wellbeing. For example, where wellbeing is more specifically fatigue, how quickly an individual completes a morning routine provides information on the fatigue level of the individual. Specific aspects of the ritual may be, for example, whether the user responds quickly to prompts on devices; how long the user takes to get out of bed (e.g., number of alarm snoozes); how long it takes the user to take a shower, brush teeth, comb hair, or perform other customary morning tasks. According to some aspects, the user can input information regarding the routine, such as how long the routine took, the subjective feeling of the user while performing the routine (e.g., tired, lagging, refreshed, excited, etc.). Alternatively, or in addition, the one or more devices and/or systems that the user interfaces with can input the information regarding the routine. The systems and devices of the present disclosure can then use the information from the morning routine, or any repeatable routine, for determining the wellbeing of the user and providing an indication of the wellbeing. Other routines can include, without limitation, games and/or tests that the user performs on a routine or semi-routine basis, such as crossword puzzles or other games that the user performs on a smart device.

According to some implementations, the systems and devices of the present disclosure can determine what within the surroundings may have contributed to changes in wellbeing. Information regarding light, sound, and/or environment can be collected and analyzed to determine why a change in wellbeing may have occurred or why a change in wellbeing has not occured. Smart devices within the surroundings of the user can collect this information. Such devices include, for example, Amazon Echo, Google Home, Nest thermostats, and other home ecosystems. Such light information may be in the form of greater ambient lighting than normal during night, which may have affected sleep. Such noise information may be in the form of greater noise than normal during the day and/or night, which may have affected sleep and/or emotional fatigue. Environmental information, such as temperature, humidity, pressure, etc., can be collected and analyzed to determine how the environment may have affected the wellbeing. For example, a hotter than average temperature at night might have affected sleep.

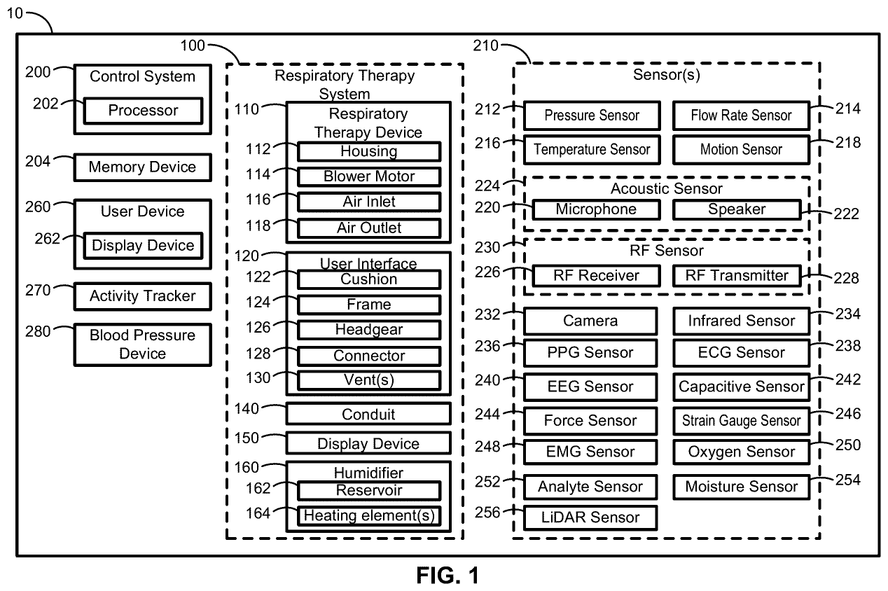

Referring to FIG. 1, a system 10, according to some implementations of the present disclosure, is illustrated. The system 10 includes a respiratory therapy system 100, a control system 200, one or more sensors 210, a user device 260, and an activity tracker 270.

The respiratory therapy system 100 includes a respiratory pressure therapy (RPT) device 110 (referred to herein as respiratory therapy device 110), a user interface 120 (also referred to as a mask or a patient interface), a conduit 140 (also referred to as a tube or an air circuit), a display device 150, and a humidifier 160. Respiratory pressure therapy refers to the application of a supply of air to an entrance to a user's airways at a controlled target pressure that is nominally positive with respect to atmosphere throughout the user's breathing cycle (e.g., in contrast to negative pressure therapies such as the tank ventilator or cuirass). The respiratory therapy system 100 is generally used to treat individuals suffering from one or more sleep-related respiratory disorders (e.g., obstructive sleep apnea, central sleep apnea, or mixed sleep apnea).

The respiratory therapy system 100 can be used, for example, as a ventilator or as a positive airway pressure (PAP) system, such as a continuous positive airway pressure (CPAP) system, an automatic positive airway pressure system (APAP), a bi-level or variable positive airway pressure system (BPAP or VPAP), or any combination thereof. The CPAP system delivers a predetermined air pressure (e.g., determined by a sleep physician) to the user. The APAP system automatically varies the air pressure delivered to the user based on, for example, respiration data associated with the user. The BPAP or VPAP system is configured to deliver a first predetermined pressure (e.g., an inspiratory positive airway pressure or IPAP) and a second predetermined pressure (e.g., an expiratory positive airway pressure or EPAP) that is lower than the first predetermined pressure.

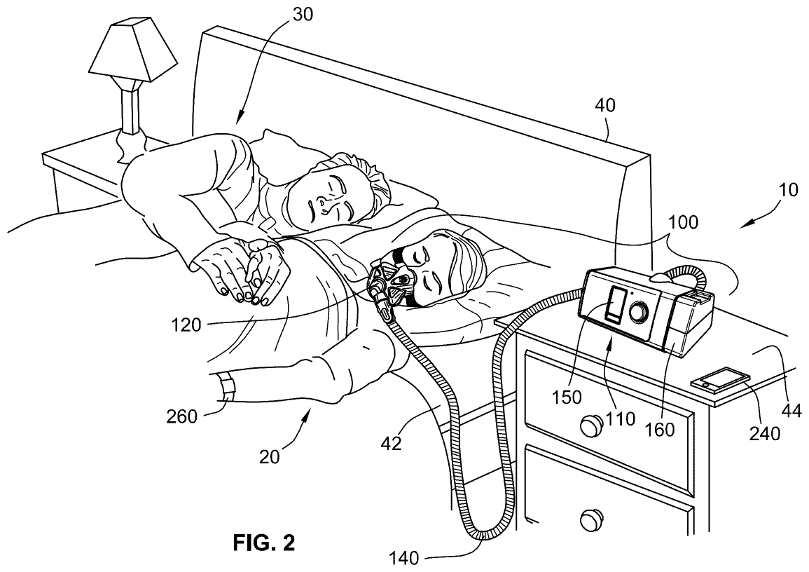

As shown in FIG. 2, the respiratory therapy system 100 can be used to treat user 20. In this example, the user 20 of the respiratory therapy system 100 and a bed partner 30 are located in a bed 40 and are laying on a mattress 42. The user interface 120 can be worn by the user 20 during a sleep session. The respiratory therapy system 100 generally aids in increasing the air pressure in the throat of the user 20 to aid in preventing the airway from closing and/or narrowing during sleep. The respiratory therapy device 110 can be positioned on a nightstand 44 that is directly adjacent to the bed 40 as shown in FIG. 2, or more generally, on any surface or structure that is generally adjacent to the bed 40 and/or the user 20.

The respiratory therapy device 110 is generally used to generate pressurized air that is delivered to a user (e.g., using one or more motors that drive one or more compressors). In some implementations, the respiratory therapy device 110 generates continuous constant air pressure that is delivered to the user. In other implementations, the respiratory therapy device 110 generates two or more predetermined pressures (e.g., a first predetermined air pressure and a second predetermined air pressure). In still other implementations, the respiratory therapy device 110 generates a variety of different air pressures within a predetermined range. For example, the respiratory therapy device 110 can deliver at least about 6 cmH₂O, at least about 10 cmH₂O, at least about 20 cmH₂O, between about 6 cmH₂O and about 10 cmH₂O, between about 7 cmH₂O and about 12 cmH₂O, etc. The respiratory therapy device 110 can also deliver pressurized air at a predetermined flow rate between, for example, about-20 L/min and about 150 L/min, while maintaining a positive pressure (relative to the ambient pressure).

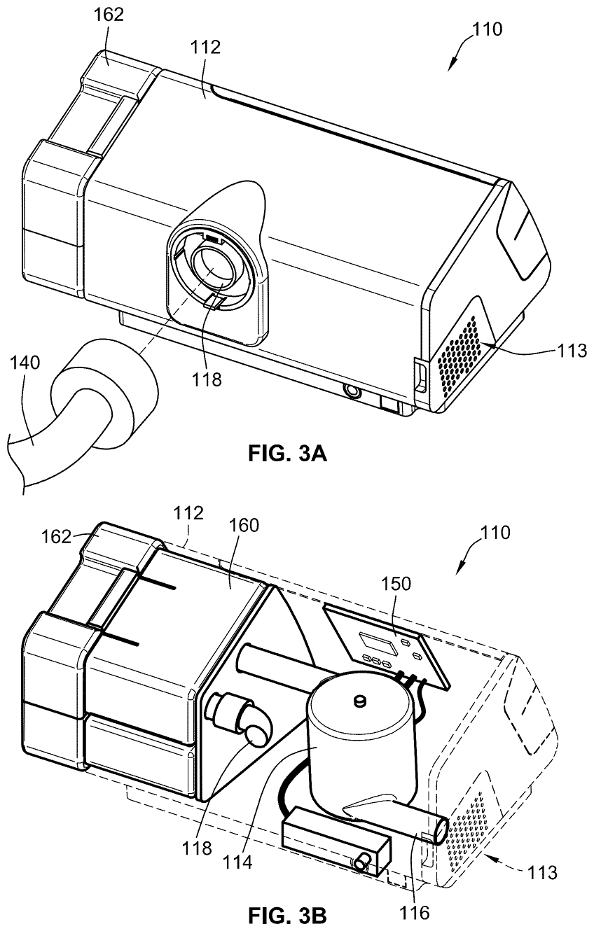

The respiratory therapy device 110 includes a housing 112, a blower motor 114, an air inlet 116, and an air outlet 118 (FIG. 1). Referring to FIGS. 3A and 3B, the blower motor 114 is at least partially disposed or integrated within the housing 112. The blower motor 114 draws air from outside the housing 112 (e.g., atmosphere) via the air inlet 116 and causes pressurized air to flow through the humidifier 160, and through the air outlet 118. In some implementations, the air inlet 116 and/or the air outlet 118 include a cover that is moveable between a closed position and an open position (e.g., to prevent or inhibit air from flowing through the air inlet 116 or the air outlet 118). As shown in FIGS. 3A and 3B, the housing 112 can include a vent 113 to allow air to pass through the housing 112 to the air inlet 116. As described below, the conduit 140 is coupled to the air outlet 118 of the respiratory therapy device 110.

Referring back to FIG. 1, the user interface 120 engages a portion of the user's face and delivers pressurized air from the respiratory therapy device 110 to the user's airway to aid in preventing the airway from narrowing and/or collapsing during sleep. This may also increase the user's oxygen intake during sleep. Generally, the user interface 120 engages the user's face such that the pressurized air is delivered to the user's airway via the user's mouth, the user's nose, or both the user's mouth and nose. Together, the respiratory therapy device 110, the user interface 120, and the conduit 140 form an air pathway fluidly coupled with an airway of the user. The pressurized air also increases the user's oxygen intake during sleep. Depending upon the therapy to be applied, the user interface 120 may form a seal, for example, with a region or portion of the user's face, to facilitate the delivery of gas at a pressure at sufficient variance with ambient pressure to effect therapy, for example, at a positive pressure of about 10 cm H₂O relative to ambient pressure. For other forms of therapy, such as the delivery of oxygen, the user interface may not include a seal sufficient to facilitate delivery to the airways of a supply of gas at a positive pressure of about 10 cmH₂O.

The user interface 120 can include, for example, a cushion 122, a frame 124, a headgear 126, connector 128, and one or more vents 130. The cushion 122 and the frame 124 define a volume of space around the mouth and/or nose of the user. When the respiratory therapy system 100 is in use, this volume space receives pressurized air (e.g., from the respiratory therapy device 110 via the conduit 140) for passage into the airway(s) of the user. The headgear 126 is generally used to aid in positioning and/or stabilizing the user interface 120 on a portion of the user (e.g., the face), and along with the cushion 122 (which, for example, can comprise silicone, plastic, foam, etc.) aids in providing a substantially air-tight seal between the user interface 120 and the user 20. In some implementations the headgear 126 includes one or more straps (e.g., including hook and loop fasteners). The connector 128 is generally used to couple (e.g., connect and fluidly couple) the conduit 140 to the cushion 122 and/or frame 124. Alternatively, the conduit 140 can be directly coupled to the cushion 122 and/or frame 124 without the connector 128. The vent 130 can be used for permitting the escape of carbon dioxide and other gases exhaled by the user 20. The user interface 120 generally can include any suitable number of vents (e.g., one, two, five, ten, etc.).

As shown in FIG. 2, in some implementations, the user interface 120 is a facial mask (e.g., a full face mask) that covers at least a portion of the nose and mouth of the user 20. Alternatively, the user interface 120 can be a nasal mask that provides air to the nose of the user or a nasal pillow mask that delivers air directly to the nostrils of the user 20. In other implementations, the user interface 120 includes a mouthpiece (e.g., a night guard mouthpiece molded to conform to the teeth of the user, a mandibular repositioning device, etc.).

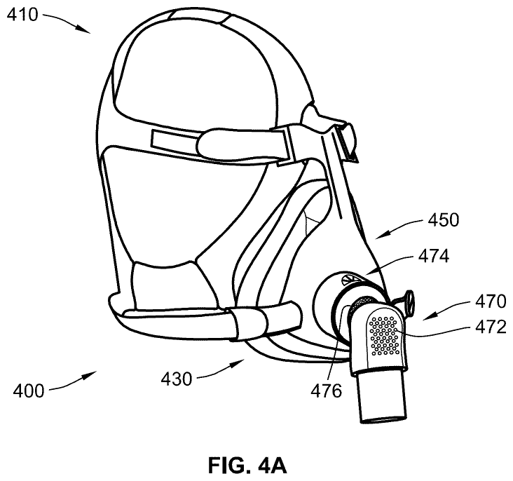

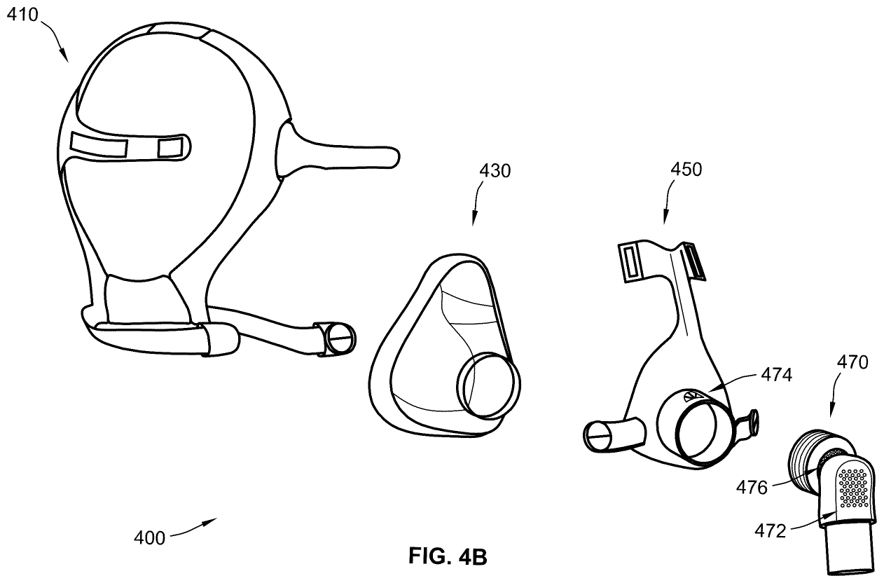

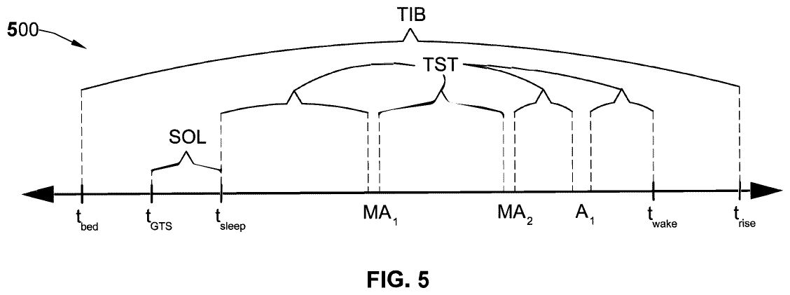

Referring to FIGS. 4A and 4B, more detailed view of one example of a user interface 400 that is the same as, or similar to, the user interface 120 (FIG. 1) according to some implementations of the present disclosure is illustrated. The user interface 400 generally includes a cushion 430 and a frame 450 that define a volume of space around the mouth and/or nose of the user. When in use, the volume of space receives pressurized air for passage into the user's airways. In some implementations, the cushion 430 and frame 450 of the user interface 400 form a unitary component of the user interface. The user interface 400 can also include a headgear 410, which generally includes a strap assembly and optionally a connector 470. The headgear 410 is configured to be positioned generally about at least a portion of a user's head when the user wears the user interface 400. The headgear 410 can be coupled to the frame 450 and positioned on the user's head such that the user's head is positioned between the headgear 410 and the frame 450. The cushion 430 is positioned between the user's face and the frame 450 to form a seal on the user's face. The optional connector 470 is configured to couple to the frame 450 and/or cushion 430 at one end and to a conduit of a respiratory therapy device (not shown). The pressurized air can flow directly from the conduit of the respiratory therapy system into the volume of space defined by the cushion 430 (or cushion 430 and frame 450) of the user interface 400 through the connector 470). From the user interface 400, the pressurized air reaches the user's airway through the user's mouth, nose, or both. Alternatively, where the user interface 400 does not include the connector 470, the conduit of the respiratory therapy system can connect directly to the cushion 430 and/or the frame 450.

In some implementations, the connector 470 may include one or more vents 472 (e.g., a plurality of vents) located on the main body of the connector 470 itself and/or one or a plurality of vents 476 (“diffuser vents”) in proximity to the frame 450, for permitting the escape of carbon dioxide (CO₂) and other gases exhaled by the user. In some implementations, one or a plurality of vents, such as vents 472 and/or 476 may be located in the user interface 400, such as in frame 450, and/or in the conduit 140. In some implementations, the frame 450 includes at least one anti-asphyxia valve (AAV) 474, which allows CO₂ and other gases exhaled by the user to escape in the event that the vents (e.g., the vents 472 or 476) fail when the respiratory therapy device is active. In general, AAVs (e.g., the AAV 474) are present for full face masks (e.g., as a safety feature); however, the diffuser vents and vents located on the mask or connector (usually an array of orifices in the mask material itself or a mesh made of some sort of fabric, in many cases replaceable) are not necessarily both present (e.g., some masks might have only the diffuser vents such as the plurality of vents 476, other masks might have only the plurality of vents 472 on the connector itself). Although the user interface 400 shown in FIGS. 4A and 4B is one example of a direct user interface, other types of user interfaces can be used, such as indirect user interfaces, and any one of the various different styles of direct and indirect user interfaces can be used according to the aspects of the present disclosure.

Referring back to FIG. 1, the conduit 140 (also referred to as an air circuit or tube) allows the flow of air between components of the respiratory therapy system 100, such as between the respiratory therapy device 110 and the user interface 120. In some implementations, there can be separate limbs of the conduit for inhalation and exhalation. In other implementations, a single limb conduit is used for both inhalation and exhalation.

Referring to FIG. 3A, the conduit 140 includes a first end 142 that is coupled to the air outlet 118 of the respiratory therapy device 110. The first end 142 can be coupled to the air outlet 118 of the respiratory therapy device 110 using a variety of techniques (e.g., a press fit connection, a snap fit connection, a threaded connection, etc.). In some implementations, the conduit 140 includes one or more heating elements that heat the pressurized air flowing through the conduit 140 (e.g., heat the air to a predetermined temperature or within a range of predetermined temperatures). Such heating elements can be coupled to and/or imbedded in the conduit 140. In such implementations, the first end 142 can include an electrical contact that is electrically coupled to the respiratory therapy device 110 to power the one or more heating elements of the conduit 140. For example, the electrical contact can be electrically coupled to an electrical contact of the air outlet 118 of the respiratory therapy device 110. In this example, electrical contact of the conduit 140 can be a male connector and the electrical contact of the air outlet 118 can be female connector, or, alternatively, the opposite configuration can be used.

The display device 150 is generally used to display image(s) including still images, video images, or both and/or information regarding the respiratory therapy device 110. For example, the display device 150 can provide information regarding the status of the respiratory therapy device 110 (e.g., whether the respiratory therapy device 110 is on/off, the pressure of the air being delivered by the respiratory therapy device 110, the temperature of the air being delivered by the respiratory therapy device 110, etc.) and/or other information (e.g., a sleep score and/or a therapy score, also referred to as a myAir™ score, such as described in WO 2016/061629 and U.S. Patent Pub. No. 2017/0311879, which are hereby incorporated by reference herein in their entireties, the current date/time, personal information for the user 20, etc.). In some implementations, the display device 150 acts as a human-machine interface (HMI) that includes a graphic user interface (GUI) configured to display the image(s) as an input interface. The display device 150 can be an LED display, an OLED display, an LCD display, or the like. The input interface can be, for example, a touchscreen or touch-sensitive substrate, a mouse, a keyboard, or any sensor system configured to sense inputs made by a human user interacting with the respiratory therapy device 110.

The humidifier 160 is coupled to or integrated in the respiratory therapy device 110 and includes a reservoir 162 for storing water that can be used to humidify the pressurized air delivered from the respiratory therapy device 110. The humidifier 160 includes a one or more heating elements 164 to heat the water in the reservoir to generate water vapor. The humidifier 160 can be fluidly coupled to a water vapor inlet of the air pathway between the blower motor 114 and the air outlet 118, or can be formed in-line with the air pathway between the blower motor 114 and the air outlet 118. For example, as shown in FIG. 3B, air flow from the air inlet 116 through the blower motor 114, and then through the humidifier 160 before exiting the respiratory therapy device 110 via the air outlet 118.

While the respiratory therapy system 100 has been described herein as including each of the respiratory therapy device 110, the user interface 120, the conduit 140, the display device 150, and the humidifier 160, more or fewer components can be included in a respiratory therapy system according to implementations of the present disclosure. For example, a first alternative respiratory therapy system includes the respiratory therapy device 110, the user interface 120, and the conduit 140. As another example, a second alternative system includes the respiratory therapy device 110, the user interface 120, and the conduit 140, and the display device 150. Thus, various respiratory therapy systems can be formed using any portion or portions of the components shown and described herein and/or in combination with one or more other components.

The control system 200 includes one or more processors 202 (hereinafter, processor 202). The control system 200 is generally used to control (e.g., actuate) the various components of the system 10 and/or analyze data obtained and/or generated by the components of the system 10. The processor 202 can be a general or special purpose processor or microprocessor. While one processor 202 is illustrated in FIG. 1, the control system 200 can include any number of processors (e.g., one processor, two processors, five processors, ten processors, etc.) that can be in a single housing, or located remotely from each other. The control system 200 (or any other control system) or a portion of the control system 200 such as the processor 202 (or any other processor(s) or portion(s) of any other control system), can be used to carry out one or more steps of any of the methods described and/or claimed herein. The control system 200 can be coupled to and/or positioned within, for example, a housing of the user device 260, a portion (e.g., the respiratory therapy device 110) of the respiratory therapy system 100, and/or within a housing of one or more of the sensors 210. The control system 200 can be centralized (within one such housing) or decentralized (within two or more of such housings, which are physically distinct). In such implementations including two or more housings containing the control system 200, the housings can be located proximately and/or remotely from each other.

The memory device 204 stores machine-readable instructions that are executable by the processor 202 of the control system 200. The memory device 204 can be any suitable computer readable storage device or media, such as, for example, a random or serial access memory device, a hard drive, a solid state drive, a flash memory device, etc. While one memory device 204 is shown in FIG. 1, the system 10 can include any suitable number of memory devices 204 (e.g., one memory device, two memory devices, five memory devices, ten memory devices, etc.). The memory device 204 can be coupled to and/or positioned within a housing of a respiratory therapy device 110 of the respiratory therapy system 100, within a housing of the user device 260, within a housing of one or more of the sensors 210, or any combination thereof. Like the control system 200, the memory device 204 can be centralized (within one such housing) or decentralized (within two or more of such housings, which are physically distinct).

In some implementations, the memory device 204 stores a user profile associated with the user. The user profile can include, for example, demographic information associated with the user, biometric information associated with the user, medical information associated with the user, self-reported user feedback, sleep parameters associated with the user (e.g., sleep-related parameters recorded from one or more earlier sleep sessions), or any combination thereof. The demographic information can include, for example, information indicative of an age of the user, a gender of the user, a race of the user, a geographic location of the user, a relationship status, a family history of insomnia or sleep apnea, an employment status of the user, an educational status of the user, a socioeconomic status of the user, or any combination thereof. The medical information can include, for example, information indicative of one or more medical conditions associated with the user, medication usage by the user, or both. The medical information data can further include a multiple sleep latency test (MSLT) result or score and/or a Pittsburgh Sleep Quality Index (PSQI) score or value. The self-reported user feedback can include information indicative of a self-reported subjective sleep score (e.g., poor, average, excellent), a self-reported subjective stress level of the user, a self-reported subjective fatigue level of the user, a self-reported subjective health status of the user, a recent life event experienced by the user, or any combination thereof.

As described herein, the processor 202 and/or memory device 204 can receive data (e.g., physiological data and/or audio data) from one or more sensors 210 (discussed further below) such that the data for storage in the memory device 204 and/or for analysis by the processor 202. The processor 202 and/or memory device 204 can communicate with the one or more sensors 210 using a wired connection or a wireless connection (e.g., using an RF communication protocol, a Wi-Fi communication protocol, a Bluetooth communication protocol, over a cellular network, etc.). In some implementations, the system 10 can include an antenna, a receiver (e.g., an RF receiver), a transmitter (e.g., an RF transmitter), a transceiver, or any combination thereof. Such components can be coupled to or integrated a housing of the control system 200 (e.g., in the same housing as the processor 202 and/or memory device 204), or the user device 260.

Referring to back to FIG. 1, the one or more sensors 210 include a pressure sensor 212, a flow rate sensor 214, a temperature sensor 216, a motion sensor 218, a microphone 220, a speaker 222, a radio-frequency (RF) receiver 226, a RF transmitter 228, a camera 232, an infrared sensor 234, a photoplethysmogram (PPG) sensor 236, an electrocardiogram (ECG) sensor 238, an electroencephalography (EEG) sensor 240, a capacitive sensor 242, a force sensor 244, a strain gauge sensor 246, an electromyography (EMG) sensor 248, an oxygen sensor 250, an analyte sensor 252, a moisture sensor 254, a LiDAR sensor 256, or any combination thereof. Generally, each of the one or more sensors 210 are configured to output sensor data that is received and stored in the memory device 204 or one or more other memory devices. Any of the one or more sensors 210 can be used to measure, detect, or otherwise collect information, such as physiological data, that can be used to determine measures of wellbeing of the user during on and/or off periods of adhering to a respiratory pressure therapy, as discussed above and below.

While the one or more sensors 210 are shown and described as including each of the pressure sensor 212, the flow rate sensor 214, the temperature sensor 216, the motion sensor 218, the microphone 220, the speaker 222, the RF receiver 226, the RF transmitter 228, the camera 232, the infrared sensor 234, the photoplethysmogram (PPG) sensor 236, the electrocardiogram (ECG) sensor 238, the electroencephalography (EEG) sensor 240, the capacitive sensor 242, the force sensor 244, the strain gauge sensor 246, the electromyography (EMG) sensor 248, the oxygen sensor 250, the analyte sensor 252, the moisture sensor 254, and the LiDAR sensor 256, more generally, the one or more sensors 210 can include any combination and any number of each of the sensors described and/or shown herein.

As described herein, the system 10 generally can be used to generate physiological data associated with a user (e.g., a user of the respiratory therapy system 100) during a sleep session. The physiological data can be analyzed to generate one or more sleep-related parameters, which can include any parameter, measurement, etc. related to the user during the sleep session. The one or more sleep-related parameters that can be determined for the user 20 during the sleep session include, for example, an Apnea-Hypopnea Index (AHI) score, a sleep score, a flow signal, a respiration signal, a respiration rate, an inspiration amplitude, an expiration amplitude, an inspiration-expiration ratio, a number of events per hour, a pattern of events, a stage, pressure settings of the respiratory therapy device 110, a heart rate, a heart rate variability, movement of the user 20, temperature, EEG activity, EMG activity, arousal, snoring, choking, coughing, whistling, wheezing, or any combination thereof.

The one or more sensors 210 can be used to generate, for example, physiological data, audio data, or both. Physiological data generated by one or more of the sensors 210 can be used by the control system 200 to determine a sleep-wake signal associated with the user 20 (FIG. 2) during the sleep session and one or more sleep-related parameters. The sleep-wake signal can be indicative of one or more sleep states, including wakefulness, relaxed wakefulness, micro-awakenings, or distinct sleep stages such as, for example, a rapid eye movement (REM) stage, a first non-REM stage (often referred to as “N1”), a second non-REM stage (often referred to as “N2”), a third non-REM stage (often referred to as “N3”), or any combination thereof. Methods for determining sleep states and/or sleep stages from physiological data generated by one or more sensors, such as the one or more sensors 210, are described in, for example, WO 2014/047310, U.S. Patent Pub. No. 2014/0088373, WO 2017/132726, WO 2019/122413, WO 2019/122414, and U.S. Patent Pub. No. 2020/0383580 each of which is hereby incorporated by reference herein in its entirety.

In some implementations, the sleep-wake signal described herein can be timestamped to indicate a time that the user enters the bed, a time that the user exits the bed, a time that the user attempts to fall asleep, etc. The sleep-wake signal can be measured by the one or more sensors 210 during the sleep session at a predetermined sampling rate, such as, for example, one sample per second, one sample per 30 seconds, one sample per minute, etc. In some implementations, the sleep-wake signal can also be indicative of a respiration signal, a respiration rate, an inspiration amplitude, an expiration amplitude, an inspiration-expiration ratio, a number of events per hour, a pattern of events, pressure settings of the respiratory therapy device 110, or any combination thereof during the sleep session. The event(s) can include snoring, apneas, central apneas, obstructive apneas, mixed apneas, hypopneas, a mask leak (e.g., from the user interface 120), a restless leg, a sleeping disorder, choking, an increased heart rate, labored breathing, an asthma attack, an epileptic episode, a seizure, or any combination thereof. The one or more sleep-related parameters that can be determined for the user during the sleep session based on the sleep-wake signal include, for example, a total time in bed, a total sleep time, a sleep onset latency, a wake-after-sleep-onset parameter, a sleep efficiency, a fragmentation index, or any combination thereof. As described in further detail herein, the physiological data and/or the sleep-related parameters can be analyzed to determine one or more sleep-related scores.

Physiological data and/or audio data generated by the one or more sensors 210 can also be used to determine a respiration signal associated with a user during a sleep session. The respiration signal is generally indicative of respiration or breathing of the user during the sleep session. The respiration signal can be indicative of and/or analyzed to determine (e.g., using the control system 200) one or more sleep-related parameters, such as, for example, a respiration rate, a respiration rate variability, an inspiration amplitude, an expiration amplitude, an inspiration-expiration ratio, an occurrence of one or more events, a number of events per hour, a pattern of events, a sleep state, a sleet stage, an apnea-hypopnea index (AHI), pressure settings of the respiratory therapy device 110, or any combination thereof. The one or more events can include snoring, apneas, central apneas, obstructive apneas, mixed apneas, hypopncas, a mask leak (e.g., from the user interface 120), a cough, a restless leg, a sleeping disorder, choking, an increased heart rate, labored breathing, an asthma attack, an epileptic episode, a seizure, increased blood pressure, or any combination thereof. Many of the described sleep-related parameters are physiological parameters, although some of the sleep-related parameters can be considered to be non-physiological parameters. Other types of physiological and/or non-physiological parameters can also be determined, either from the data from the one or more sensors 210, or from other types of data.

The pressure sensor 212 outputs pressure data that can be stored in the memory device 204 and/or analyzed by the processor 202 of the control system 200. In some implementations, the pressure sensor 212 is an air pressure sensor (e.g., barometric pressure sensor) that generates sensor data indicative of the respiration (e.g., inhaling and/or exhaling) of the user of the respiratory therapy system 100 and/or ambient pressure. In such implementations, the pressure sensor 212 can be coupled to or integrated in the respiratory therapy device 110. The pressure sensor 212 can be, for example, a capacitive sensor, an electromagnetic sensor, a piezoelectric sensor, a strain-gauge sensor, an optical sensor, a potentiometric sensor, or any combination thereof.

The flow rate sensor 214 outputs flow rate data that can be stored in the memory device 204 and/or analyzed by the processor 202 of the control system 200. Examples of flow rate sensors (such as, for example, the flow rate sensor 214) are described in International Publication No. WO 2012/012835 and U.S. Pat. No. 10,328,219, both of which are hereby incorporated by reference herein in their entireties. In some implementations, the flow rate sensor 214 is used to determine an air flow rate from the respiratory therapy device 110, an air flow rate through the conduit 140, an air flow rate through the user interface 120, or any combination thereof. In such implementations, the flow rate sensor 214 can be coupled to or integrated in the respiratory therapy device 110, the user interface 120, or the conduit 140. The flow rate sensor 214 can be a mass flow rate sensor such as, for example, a rotary flow meter (e.g., Hall effect flow meters), a turbine flow meter, an orifice flow meter, an ultrasonic flow meter, a hot wire sensor, a vortex sensor, a membrane sensor, or any combination thereof. In some implementations, the flow rate sensor 214 is configured to measure a vent flow (e.g., intentional “leak”), an unintentional leak (e.g., mouth leak and/or mask leak), a patient flow (e.g., air into and/or out of lungs), or any combination thereof. In some implementations, the flow rate data can be analyzed to determine cardiogenic oscillations of the user. In some examples, the pressure sensor 212 can be used to determine a blood pressure of a user.

The temperature sensor 216 outputs temperature data that can be stored in the memory device 204 and/or analyzed by the processor 202 of the control system 200. In some implementations, the temperature sensor 216 generates temperatures data indicative of a core body temperature of the user 20 (FIG. 2), a skin temperature of the user 20, a temperature of the air flowing from the respiratory therapy device 110 and/or through the conduit 140, a temperature in the user interface 120, an ambient temperature, or any combination thereof. The temperature sensor 216 can be, for example, a thermocouple sensor, a thermistor sensor, a silicon band gap temperature sensor or semiconductor-based sensor, a resistance temperature detector, or any combination thereof.

The motion sensor 218 outputs motion data that can be stored in the memory device 204 and/or analyzed by the processor 202 of the control system 200. The motion sensor 218 can be used to detect movement of the user 20 during the sleep session, and/or detect movement of any of the components of the respiratory therapy system 100, such as the respiratory therapy device 110, the user interface 120, or the conduit 140. The motion sensor 218 can include one or more inertial sensors, such as accelerometers, gyroscopes, and magnetometers. In some implementations, the motion sensor 218 alternatively or additionally generates one or more signals representing bodily movement of the user, from which may be obtained a signal representing a sleep state of the user; for example, via a respiratory movement of the user. In some implementations, the motion data from the motion sensor 218 can be used in conjunction with additional data from another one of the sensors 210 to determine the sleep state of the user.

The microphone 220 outputs sound and/or audio data that can be stored in the memory device 204 and/or analyzed by the processor 202 of the control system 200. The audio data generated by the microphone 220 is reproducible as one or more sound(s) during a sleep session (e.g., sounds from the user 20). The audio data form the microphone 220 can also be used to identify (e.g., using the control system 200) an event experienced by the user during the sleep session, as described in further detail herein. The microphone 220 can be coupled to or integrated in the respiratory therapy device 110, the user interface 120, the conduit 140, or the user device 260. In some implementations, the system 10 includes a plurality of microphones (e.g., two or more microphones and/or an array of microphones with beamforming) such that sound data generated by each of the plurality of microphones can be used to discriminate the sound data generated by another of the plurality of microphones

The speaker 222 outputs sound waves that are audible to a user of the system 10 (e.g., the user 20 of FIG. 2). The speaker 222 can be used, for example, as an alarm clock or to play an alert or message to the user 20 (e.g., in response to an event). In some implementations, the speaker 222 can be used to communicate the audio data generated by the microphone 220 to the user. The speaker 222 can be coupled to or integrated in the respiratory therapy device 110, the user interface 120, the conduit 140, or the user device 260.

The microphone 220 and the speaker 222 can be used as separate devices. In some implementations, the microphone 220 and the speaker 222 can be combined into an acoustic sensor 224 (e.g., a SONAR sensor), as described in, for example, WO 2018/050913, WO 2020/104465, U.S. Pat. App. Pub. No. 2022/0007965, each of which is hereby incorporated by reference herein in its entirety. In such implementations, the speaker 222 generates or emits sound waves at a predetermined interval and the microphone 220 detects the reflections of the emitted sound waves from the speaker 222. The sound waves generated or emitted by the speaker 222 have a frequency that is not audible to the human car (e.g., below 20 Hz or above around 18 kHz) so as not to disturb the sleep of the user 20 or the bed partner 30 (FIG. 2). Based at least in part on the data from the microphone 220 and/or the speaker 222, the control system 200 can determine a location of the user 20 (FIG. 2) and/or one or more of the sleep-related parameters described in herein such as, for example, a respiration signal, a respiration rate, an inspiration amplitude, an expiration amplitude, an inspiration-expiration ratio, a number of events per hour, a pattern of events, a sleep state, a sleep stage, pressure settings of the respiratory therapy device 110, or any combination thereof. In such a context, a sonar sensor may be understood to concern an active acoustic sensing, such as by generating and/or transmitting ultrasound and/or low frequency ultrasound sensing signals (e.g., in a frequency range of about 17-23 kHz, 18-22 kHz, or 17-18 kHz, for example), through the air.

In some implementations, the sensors 210 include (i) a first microphone that is the same as, or similar to, the microphone 220, and is integrated in the acoustic sensor 224 and (ii) a second microphone that is the same as, or similar to, the microphone 220, but is separate and distinct from the first microphone that is integrated in the acoustic sensor 224.

The RF transmitter 228 generates and/or emits radio waves having a predetermined frequency and/or a predetermined amplitude (e.g., within a high frequency band, within a low frequency band, long wave signals, short wave signals, etc.). The RF receiver 226 detects the reflections of the radio waves emitted from the RF transmitter 228, and this data can be analyzed by the control system 200 to determine a location of the user and/or one or more of the sleep-related parameters described herein. An RF receiver (either the RF receiver 226 and the RF transmitter 228 or another RF pair) can also be used for wireless communication between the control system 200, the respiratory therapy device 110, the one or more sensors 210, the user device 260, or any combination thereof. While the RF receiver 226 and RF transmitter 228 are shown as being separate and distinct elements in FIG. 1, in some implementations, the RF receiver 226 and RF transmitter 228 are combined as a part of an RF sensor 230 (e.g. a RADAR sensor). In some such implementations, the RF sensor 230 includes a control circuit. The format of the RF communication can be Wi-Fi, Bluetooth, or the like.

In some implementations, the RF sensor 230 is a part of a mesh system. One example of a mesh system is a Wi-Fi mesh system, which can include mesh nodes, mesh router(s), and mesh gateway(s), each of which can be mobile/movable or fixed. In such implementations, the Wi-Fi mesh system includes a Wi-Fi router and/or a Wi-Fi controller and one or more satellites (e.g., access points), each of which include an RF sensor that the is the same as, or similar to, the RF sensor 230. The Wi-Fi router and satellites continuously communicate with one another using Wi-Fi signals. The Wi-Fi mesh system can be used to generate motion data based on changes in the Wi-Fi signals (e.g., differences in received signal strength) between the router and the satellite(s) due to an object or person moving partially obstructing the signals. The motion data can be indicative of motion, breathing, heart rate, gait, falls, behavior, etc., or any combination thereof.

The camera 232 outputs image data reproducible as one or more images (e.g., still images, video images, thermal images, or any combination thereof) that can be stored in the memory device 204. The image data from the camera 232 can be used by the control system 200 to determine one or more of the sleep-related parameters described herein, such as, for example, one or more events (e.g., periodic limb movement or restless leg syndrome), a respiration signal, a respiration rate, an inspiration amplitude, an expiration amplitude, an inspiration-expiration ratio, a number of events per hour, a pattern of events, a sleep state, a sleep stage, or any combination thereof. Further, the image data from the camera 232 can be used to, for example, identify a location of the user, to determine chest movement of the user (FIG. 2), to determine air flow of the mouth and/or nose of the user, to determine a time when the user enters the bed (FIG. 2), and to determine a time when the user exits the bed. In some implementations, the camera 232 includes a wide angle lens or a fish eye lens.

The infrared (IR) sensor 234 outputs infrared image data reproducible as one or more infrared images (e.g., still images, video images, or both) that can be stored in the memory device 204. The infrared data from the IR sensor 234 can be used to determine one or more sleep-related parameters during a sleep session, including a temperature of the user 20 and/or movement of the user 20. The IR sensor 234 can also be used in conjunction with the camera 232 when measuring the presence, location, and/or movement of the user 20. The IR sensor 234 can detect infrared light having a wavelength between about 700 nm and about 1 mm, for example, while the camera 232 can detect visible light having a wavelength between about 380 nm and about 740 nm.

The PPG sensor 236 outputs physiological data associated with the user 20 (FIG. 2) that can be used to determine one or more sleep-related parameters, such as, for example, a heart rate, a heart rate variability, a cardiac cycle, respiration rate, an inspiration amplitude, an expiration amplitude, an inspiration-expiration ratio, estimated blood pressure parameter(s), or any combination thereof. The PPG sensor 236 can be worn by the user 20, embedded in clothing and/or fabric that is worn by the user 20, embedded in and/or coupled to the user interface 120 and/or its associated headgear (e.g., straps, etc.), etc.

The ECG sensor 238 outputs physiological data associated with electrical activity of the heart of the user 20. In some implementations, the ECG sensor 238 includes one or more electrodes that are positioned on or around a portion of the user 20 during the sleep session. The physiological data from the ECG sensor 238 can be used, for example, to determine one or more of the sleep-related parameters described herein.

The EEG sensor 240 outputs physiological data associated with electrical activity of the brain of the user 20. In some implementations, the EEG sensor 240 includes one or more electrodes that are positioned on or around the scalp of the user 20 during the sleep session. The physiological data from the EEG sensor 240 can be used, for example, to determine a sleep state and/or a sleep stage of the user 20 at any given time during the sleep session. In some implementations, the EEG sensor 240 can be integrated in the user interface 120 and/or the associated headgear (e.g., straps, etc.).

The capacitive sensor 242, the force sensor 244, and the strain gauge sensor 246 output data that can be stored in the memory device 204 and used/analyzed by the control system 200 to determine, for example, one or more of the sleep-related parameters described herein. The EMG sensor 248 outputs physiological data associated with electrical activity produced by one or more muscles. The oxygen sensor 250 outputs oxygen data indicative of an oxygen concentration of gas (e.g., in the conduit 140 or at the user interface 120). The oxygen sensor 250 can be, for example, an ultrasonic oxygen sensor, an electrical oxygen sensor, a chemical oxygen sensor, an optical oxygen sensor, a pulse oximeter (e.g., SpO₂ sensor), or any combination thereof.

The analyte sensor 252 can be used to detect the presence of an analyte in the exhaled breath of the user 20. The data output by the analyte sensor 252 can be stored in the memory device 204 and used by the control system 200 to determine the identity and concentration of any analytes in the breath of the user. In some implementations, the analyte sensor 174 is positioned near a mouth of the user to detect analytes in breath exhaled from the user's mouth. For example, when the user interface 120 is a facial mask that covers the nose and mouth of the user, the analyte sensor 252 can be positioned within the facial mask to monitor the user's mouth breathing. In other implementations, such as when the user interface 120 is a nasal mask or a nasal pillow mask, the analyte sensor 252 can be positioned near the nose of the user to detect analytes in breath exhaled through the user's nose. In still other implementations, the analyte sensor 252 can be positioned near the user's mouth when the user interface 120 is a nasal mask or a nasal pillow mask. In this implementation, the analyte sensor 252 can be used to detect whether any air is inadvertently leaking from the user's mouth and/or the user interface 120. In some implementations, the analyte sensor 252 is a volatile organic compound (VOC) sensor that can be used to detect carbon-based chemicals or compounds. In some implementations, the analyte sensor 174 can also be used to detect whether the user is breathing through their nose or mouth. For example, if the data output by an analyte sensor 252 positioned near the mouth of the user or within the facial mask (e.g., in implementations where the user interface 120 is a facial mask) detects the presence of an analyte, the control system 200 can use this data as an indication that the user is breathing through their mouth.

The moisture sensor 254 outputs data that can be stored in the memory device 204 and used by the control system 200. The moisture sensor 254 can be used to detect moisture in various areas surrounding the user (e.g., inside the conduit 140 or the user interface 120, near the user's face, near the connection between the conduit 140 and the user interface 120, near the connection between the conduit 140 and the respiratory therapy device 110, etc.). Thus, in some implementations, the moisture sensor 254 can be coupled to or integrated in the user interface 120 or in the conduit 140 to monitor the humidity of the pressurized air from the respiratory therapy device 110. In other implementations, the moisture sensor 254 is placed near any area where moisture levels need to be monitored. The moisture sensor 254 can also be used to monitor the humidity of the ambient environment surrounding the user, for example, the air inside the bedroom.

The Light Detection and Ranging (LiDAR) sensor 256 can be used for depth sensing. This type of optical sensor (e.g., laser sensor) can be used to detect objects and build three dimensional (3D) maps of the surroundings, such as of a living space. LiDAR can generally utilize a pulsed laser to make time of flight measurements. LiDAR is also referred to as 3D laser scanning. In an example of use of such a sensor, a fixed or mobile device (such as a smartphone) having a LiDAR sensor 256 can measure and map an area extending 5 meters or more away from the sensor. The LiDAR data can be fused with point cloud data estimated by an electromagnetic RADAR sensor, for example. The LiDAR sensor(s) 256 can also use artificial intelligence (AI) to automatically geofence RADAR systems by detecting and classifying features in a space that might cause issues for RADAR systems, such a glass windows (which can be highly reflective to RADAR). LiDAR can also be used to provide an estimate of the height of a person, as well as changes in height when the person sits down, or falls down, for example. LiDAR may be used to form a 3D mesh representation of an environment. In a further use, for solid surfaces through which radio waves pass (e.g., radio-translucent materials), the LiDAR may reflect off such surfaces, thus allowing a classification of different type of obstacles.

In some implementations, the one or more sensors 210 also include a galvanic skin response (GSR) sensor, a blood flow sensor, a respiration sensor, a pulse sensor, a sphygmomanometer sensor, an oximetry sensor, a sonar sensor, a RADAR sensor, a blood glucose sensor, a color sensor, a pH sensor, an air quality sensor, a tilt sensor, a rain sensor, a soil moisture sensor, a water flow sensor, an alcohol sensor, or any combination thereof.

While shown separately in FIG. 1, any combination of the one or more sensors 210 can be integrated in and/or coupled to any one or more of the components of the system 100, including the respiratory therapy device 110, the user interface 120, the conduit 140, the humidifier 160, the control system 200, the user device 260, the activity tracker 270, or any combination thereof. For example, the microphone 220 and the speaker 222 can be integrated in and/or coupled to the user device 260 and the pressure sensor 212 and/or flow rate sensor 132 are integrated in and/or coupled to the respiratory therapy device 110. In some implementations, at least one of the one or more sensors 210 is not coupled to the respiratory therapy device 110, the control system 200, or the user device 260, and is positioned generally adjacent to the user 20 during the sleep session (e.g., positioned on or in contact with a portion of the user 20, worn by the user 20, coupled to or positioned on the nightstand, coupled to the mattress, coupled to the ceiling, etc.).

One or more of the respiratory therapy device 110, the user interface 120, the conduit 140, the display device 150, and the humidifier 160 can contain one or more sensors (e.g., a pressure sensor, a flow rate sensor, or more generally any of the other sensors 210 described herein). These one or more sensors can be used, for example, to measure the air pressure and/or flow rate of pressurized air supplied by the respiratory therapy device 110.

The data from the one or more sensors 210 can be analyzed (e.g., by the control system 200) to determine one or more sleep-related parameters, which can include a respiration signal, a respiration rate, a respiration pattern, an inspiration amplitude, an expiration amplitude, an inspiration-expiration ratio, an occurrence of one or more events, a number of events per hour, a pattern of events, a sleep state, an apnea-hypopnea index (AHI), or any combination thereof. The one or more events can include snoring, apneas, central apneas, obstructive apneas, mixed apneas, hypopneas, a mask leak, a cough, a restless leg, a sleeping disorder, choking, an increased heart rate, labored breathing, an asthma attack, an epileptic episode, a seizure, increased blood pressure, or any combination thereof. Many of these sleep-related parameters are physiological parameters, although some of the sleep-related parameters can be considered to be non-physiological parameters. Other types of physiological and non-physiological parameters can also be determined, either from the data from the one or more sensors 210, or from other types of data.

The user device 260 (FIG. 1) includes a display device 262. The user device 260 can be, for example, a mobile device such as a smart phone, a tablet, a gaming console, a smart watch, a laptop, or the like. Alternatively, the user device 260 can be an external sensing system, a television (e.g., a smart television) or another smart home device (e.g., a smart speaker(s) such as Google Home, Amazon Echo, Alexa etc.). In some implementations, the user device is a wearable device (e.g., a smart watch). The display device 262 is generally used to display image(s) including still images, video images, or both. In some implementations, the display device 262 acts as a human-machine interface (HMI) that includes a graphic user interface (GUI) configured to display the image(s) and an input interface. The display device 262 can be an LED display, an OLED display, an LCD display, or the like. The input interface can be, for example, a touchscreen or touch-sensitive substrate, a mouse, a keyboard, or any sensor system configured to sense inputs made by a human user interacting with the user device 260. In some implementations, one or more user devices can be used by and/or included in the system 10.

In some implementations, the system 100 also includes an activity tracker 270. The activity tracker 270 is generally used to aid in generating physiological data associated with the user. The activity tracker 270 can include one or more of the sensors 210 described herein, such as, for example, the motion sensor 138 (e.g., one or more accelerometers and/or gyroscopes), the PPG sensor 154, and/or the ECG sensor 156. The physiological data from the activity tracker 270 can be used to determine, for example, a number of steps, a distance traveled, a number of steps climbed, a duration of physical activity, a type of physical activity, an intensity of physical activity, time spent standing, a respiration rate, an average respiration rate, a resting respiration rate, a maximum he respiration art rate, a respiration rate variability, a heart rate, an average heart rate, a resting heart rate, a maximum heart rate, a heart rate variability, a number of calories burned, blood oxygen saturation, electrodermal activity (also known as skin conductance or galvanic skin response), or any combination thereof. In some implementations, the activity tracker 270 is coupled (e.g., electronically or physically) to the user device 260.

In some implementations, the activity tracker 270 is a wearable device that can be worn by the user, such as a smartwatch, a wristband, a ring, or a patch. For example, referring to FIG. 2, the activity tracker 270 is worn on a wrist of the user 20. The activity tracker 270 can also be coupled to or integrated a garment or clothing that is worn by the user. Alternatively still, the activity tracker 270 can also be coupled to or integrated in (e.g., within the same housing) the user device 260. More generally, the activity tracker 270 can be communicatively coupled with, or physically integrated in (e.g., within a housing), the control system 200, the memory device 204, the respiratory therapy system 100, and/or the user device 260.

In some implementations, the system 100 also includes a blood pressure device 280. The blood pressure device 280 is generally used to aid in generating cardiovascular data for determining one or more blood pressure measurements associated with the user 20. The blood pressure device 280 can include at least one of the one or more sensors 210 to measure, for example, a systolic blood pressure component and/or a diastolic blood pressure component.

In some implementations, the blood pressure device 280 is a sphygmomanometer including an inflatable cuff that can be worn by the user 20 and a pressure sensor (e.g., the pressure sensor 212 described herein). For example, in the example of FIG. 2, the blood pressure device 280 can be worn on an upper arm of the user 20. In such implementations where the blood pressure device 280 is a sphygmomanometer, the blood pressure device 280 also includes a pump (e.g., a manually operated bulb) for inflating the cuff. In some implementations, the blood pressure device 280 is coupled to the respiratory therapy device 110 of the respiratory therapy system 100, which in turn delivers pressurized air to inflate the cuff. More generally, the blood pressure device 280 can be communicatively coupled with, and/or physically integrated in (e.g., within a housing), the control system 200, the memory device 204, the respiratory therapy system 100, the user device 260, and/or the activity tracker 270.

In other implementations, the blood pressure device 280 is an ambulatory blood pressure monitor communicatively coupled to the respiratory therapy system 100. An ambulatory blood pressure monitor includes a portable recording device attached to a belt or strap worn by the user 20 and an inflatable cuff attached to the portable recording device and worn around an arm of the user 20. The ambulatory blood pressure monitor is configured to measure blood pressure between about every fifteen minutes to about thirty minutes over a 24-hour or a 48-hour period. The ambulatory blood pressure monitor may measure heart rate of the user 20 at the same time. These multiple readings are averaged over the 24-hour period. The ambulatory blood pressure monitor determines any changes in the measured blood pressure and heart rate of the user 20, as well as any distribution and/or trending patterns of the blood pressure and heart rate data during a sleeping period and an awakened period of the user 20. The measured data and statistics may then be communicated to the respiratory therapy system 100.

The blood pressure device 280 maybe positioned external to the respiratory therapy system 100, coupled directly or indirectly to the user interface 120, coupled directly or indirectly to a headgear associated with the user interface 120, or inflatably coupled to or about a portion of the user 20. The blood pressure device 280 is generally used to aid in generating physiological data for determining one or more blood pressure measurements associated with a user, for example, a systolic blood pressure component and/or a diastolic blood pressure component. In some implementations, the blood pressure device 280 is a sphygmomanometer including an inflatable cuff that can be worn by a user and a pressure sensor (e.g., the pressure sensor 212 described herein).

In some implementations, the blood pressure device 280 is an invasive device which can continuously monitor arterial blood pressure of the user 20 and take an arterial blood sample on demand for analyzing gas of the arterial blood. In some other implementations, the blood pressure device 280 is a continuous blood pressure monitor, using a radio frequency sensor and capable of measuring blood pressure of the user 20 once very few seconds (e.g., every 3 seconds, every 5 seconds, every 7 seconds, etc.) The radio frequency sensor may use continuous wave, frequency-modulated continuous wave (FMCW with ramp chirp, triangle, sinewave), other schemes such as PSK, FSK etc., pulsed continuous wave, and/or spread in ultra wideband ranges (which may include spreading, PRN codes or impulse systems).

While the control system 200 and the memory device 204 are described and shown in FIG. 1 as being a separate and distinct component of the system 100, in some implementations, the control system 200 and/or the memory device 204 are integrated in the user device 260 and/or the respiratory therapy device 110. Alternatively, in some implementations, the control system 200 or a portion thereof (e.g., the processor 202) can be located in a cloud (e.g., integrated in a server, integrated in an Internet of Things (IoT) device, connected to the cloud, be subject to edge cloud processing, etc.), located in one or more servers (e.g., remote servers, local servers, etc., or any combination thereof.

While system 100 is shown as including all of the components described above, more or fewer components can be included in a system according to implementations of the present disclosure. For example, a first alternative system includes the control system 200, the memory device 204, and at least one of the one or more sensors 210 and does not include the respiratory therapy system 100. As another example, a second alternative system includes the control system 200, the memory device 204, at least one of the one or more sensors 210, and the user device 260. As yet another example, a third alternative system includes the control system 200, the memory device 204, the respiratory therapy system 100, at least one of the one or more sensors 210, and the user device 260. Thus, various systems can be formed using any portion or portions of the components shown and described herein and/or in combination with one or more other components.

As used herein, a sleep session can be defined in multiple ways. For example, a sleep session can be defined by an initial start time and an end time. In some implementations, a sleep session is a duration where the user is asleep, that is, the sleep session has a start time and an end time, and during the sleep session, the user does not wake until the end time. That is, any period of the user being awake is not included in a sleep session. From this first definition of sleep session, if the user wakes ups and falls asleep multiple times in the same night, each of the sleep intervals separated by an awake interval is a sleep session.

Alternatively, in some implementations, a sleep session has a start time and an end time, and during the sleep session, the user can wake up, without the sleep session ending, so long as a continuous duration that the user is awake is below an awake duration threshold. The awake duration threshold can be defined as a percentage of a sleep session. The awake duration threshold can be, for example, about twenty percent of the sleep session, about fifteen percent of the sleep session duration, about ten percent of the sleep session duration, about five percent of the sleep session duration, about two percent of the sleep session duration, etc., or any other threshold percentage. In some implementations, the awake duration threshold is defined as a fixed amount of time, such as, for example, about one hour, about thirty minutes, about fifteen minutes, about ten minutes, about five minutes, about two minutes, etc., or any other amount of time.

In some implementations, a sleep session is defined as the entire time between the time in the evening at which the user first entered the bed, and the time the next morning when user last left the bed. Put another way, a sleep session can be defined as a period of time that begins on a first date (e.g., Monday, Jan. 6, 2020) at a first time (e.g., 10:00 PM), that can be referred to as the current evening, when the user first enters a bed with the intention of going to sleep (e.g., not if the user intends to first watch television or play with a smart phone before going to sleep, etc.), and ends on a second date (e.g., Tuesday, Jan. 7, 2020) at a second time (e.g., 7:00 AM), that can be referred to as the next morning, when the user first exits the bed with the intention of not going back to sleep that next morning.

In some implementations, the user can manually define the beginning of a sleep session and/or manually terminate a sleep session. For example, the user can select (e.g., by clicking or tapping) one or more user-selectable element that is displayed on the display device 262 of the user device 260 (FIG. 1) to manually initiate or terminate the sleep session.

Generally, the sleep session includes any point in time after the user 20 has laid or sat down in the bed 40 (or another area or object on which they intend to sleep), and has turned on the respiratory therapy device 110 and donned the user interface 120. The sleep session can thus include time periods (i) when the user 20 is using the respiratory therapy system 100, but before the user 20 attempts to fall asleep (for example when the user 20 lays in the bed 40 reading a book); (ii) when the user 20 begins trying to fall asleep but is still awake; (iii) when the user 20 is in a light sleep (also referred to as stage 1 and stage 2 of non-rapid eye movement (NREM) sleep); (iv) when the user 20 is in a deep sleep (also referred to as slow-wave sleep, SWS, or stage 3 of NREM sleep); (v) when the user 20 is in rapid eye movement (REM) sleep; (vi) when the user 20 is periodically awake between light sleep, deep sleep, or REM sleep; or (vii) when the user 20 wakes up and does not fall back asleep.

The sleep session is generally defined as ending once the user 20 removes the user interface 120, turns off the respiratory therapy device 110, and gets out of bed 40. In some implementations, the sleep session can include additional periods of time, or can be limited to only some of the above-disclosed time periods. For example, the sleep session can be defined to encompass a period of time beginning when the respiratory therapy device 110 begins supplying the pressurized air to the airway or the user 20, ending when the respiratory therapy device 110 stops supplying the pressurized air to the airway of the user 20, and including some or all of the time points in between, when the user 20 is asleep or awake.

Referring to the timeline 500 in FIG. 5 the enter bed time t_{bed} is associated with the time that the user initially enters the bed (e.g., bed 40 in FIG. 2) prior to falling asleep (e.g., when the user lies down or sits in the bed). The enter bed time t_{bed} can be identified based on a bed threshold duration to distinguish between times when the user enters the bed for sleep and when the user enters the bed for other reasons (e.g., to watch TV). For example, the bed threshold duration can be at least about 10 minutes, at least about 20 minutes, at least about 30 minutes, at least about 45 minutes, at least about 1 hour, at least about 2 hours, etc. While the enter bed time t_{bed} is described herein in reference to a bed, more generally, the enter time t_{bed} can refer to the time the user initially enters any location for sleeping (e.g., a couch, a chair, a sleeping bag, etc.).

The go-to-sleep time (GTS) is associated with the time that the user initially attempts to fall asleep after entering the bed (t_{bed}). For example, after entering the bed, the user may engage in one or more activities to wind down prior to trying to sleep (e.g., reading, watching TV, listening to music, using the user device 260, etc.). The initial sleep time (tₛₗₑₑₚ) is the time that the user initially falls asleep. For example, the initial sleep time (tₛₗₑₑₚ) can be the time that the user initially enters the first non-REM sleep stage.

The wake-up time t_{wake} is the time associated with the time when the user wakes up without going back to sleep (e.g., as opposed to the user waking up in the middle of the night and going back to sleep). The user may experience one of more unconscious microawakenings (e.g., microawakenings MA₁ and MA₂) having a short duration (e.g., 5 seconds, 10 seconds, 30 seconds, 1 minute, etc.) after initially falling asleep. In contrast to the wake-up time t_{wake}, the user goes back to sleep after each of the microawakenings MA₁ and MA₂. Similarly, the user may have one or more conscious awakenings (e.g., awakening A) after initially falling asleep (e.g., getting up to go to the bathroom, attending to children or pets, sleep walking, etc.). However, the user goes back to sleep after the awakening A. Thus, the wake-up time t_{wake} can be defined, for example, based on a wake threshold duration (e.g., the user is awake for at least 15 minutes, at least 20 minutes, at least 30 minutes, at least 1 hour, etc.).

Similarly, the rising time tᵣᵢₛₑ is associated with the time when the user exits the bed and stays out of the bed with the intent to end the sleep session (e.g., as opposed to the user getting up during the night to go to the bathroom, to attend to children or pets, sleep walking, etc.). In other words, the rising time tᵣᵢₛₑ is the time when the user last leaves the bed without returning to the bed until a next sleep session (e.g., the following evening). Thus, the rising time tᵣᵢₛₑ can be defined, for example, based on a rise threshold duration (e.g., the user has left the bed for at least 15 minutes, at least 20 minutes, at least 30 minutes, at least 1 hour, etc.). The enter bed time t_{bed} time for a second, subsequent sleep session can also be defined based on a rise threshold duration (e.g., the user has left the bed for at least 4 hours, at least 6 hours, at least 8 hours, at least 12 hours, etc.).

As described above, the user may wake up and get out of bed one more times during the night between the initial t_{bed} and the final tᵣᵢₛₑ. In some implementations, the final wake-up time t_{wake} and/or the final rising time tᵣᵢₛₑ that are identified or determined based on a predetermined threshold duration of time subsequent to an event (e.g., falling asleep or leaving the bed). Such a threshold duration can be customized for the user. For a standard user which goes to bed in the evening, then wakes up and goes out of bed in the morning any period (between the user waking up (t_{wake}) or raising up (tᵣᵢₛₑ), and the user either going to bed (t_{bed}), going to sleep (t_{GTS}) or falling asleep (tₛₗₑₑₚ) of between about 12 and about 18 hours can be used. For users that spend longer periods of time in bed, shorter threshold periods may be used (e.g., between about 8 hours and about 14 hours). The threshold period may be initially selected and/or later adjusted based on the system monitoring the user's sleep behavior.

The total time in bed (TIB) is the duration of time between the time enter bed time t_{bed} and the rising time tᵣᵢₛₑ. The total sleep time (TST) is associated with the duration between the initial sleep time and the wake-up time, excluding any conscious or unconscious awakenings and/or micro-awakenings therebetween. Generally, the total sleep time (TST) will be shorter than the total time in bed (TIB) (e.g., one minute short, ten minutes shorter, one hour shorter, etc.). For example, referring to the timeline 500 of FIG. 5, the total sleep time (TST) spans between the initial sleep time tₛₗₑₑₚ and the wake-up time t_{wake}, but excludes the duration of the first micro-awakening MA₁, the second micro-awakening MA₂, and the awakening A. As shown, in this example, the total sleep time (TST) is shorter than the total time in bed (TIB).

In some implementations, the total sleep time (TST) can be defined as a persistent total sleep time (PTST). In such implementations, the persistent total sleep time excludes a predetermined initial portion or period of the first non-REM stage (e.g., light sleep stage). For example, the predetermined initial portion can be between about 30 seconds and about 20 minutes, between about 1 minute and about 10 minutes, between about 3 minutes and about 5 minutes, etc. The persistent total sleep time is a measure of sustained sleep, and smooths the sleep-wake hypnogram. For example, when the user is initially falling asleep, the user may be in the first non-REM stage for a very short time (e.g., about 30 seconds), then back into the wakefulness stage for a short period (e.g., one minute), and then goes back to the first non-REM stage. In this example, the persistent total sleep time excludes the first instance (e.g., about 30 seconds) of the first non-REM stage.

In some implementations, the sleep session is defined as starting at the enter bed time (t_{bed}) and ending at the rising time (tᵣᵢₛₑ), i.e., the sleep session is defined as the total time in bed (TIB). In some implementations, a sleep session is defined as starting at the initial sleep time (tₛₗₑₑₚ) and ending at the wake-up time (t_{wake}). In some implementations, the sleep session is defined as the total sleep time (TST). In some implementations, a sleep session is defined as starting at the go-to-sleep time (t_{GTS}) and ending at the wake-up time (t_{wake}). In some implementations, a sleep session is defined as starting at the go-to-sleep time (t_{GTS}) and ending at the rising time (tᵣᵢₛₑ). In some implementations, a sleep session is defined as starting at the enter bed time (t_{bed}) and ending at the wake-up time (t_{wake}). In some implementations, a sleep session is defined as starting at the initial sleep time (tₛₗₑₑₚ) and ending at the rising time (tᵣᵢₛₑ).

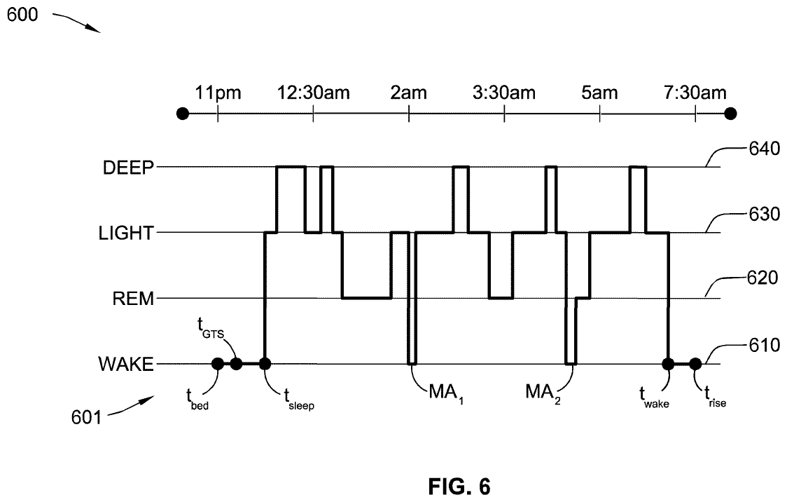

Referring to FIG. 6, an exemplary hypnogram 600 corresponding to the timeline 500 (FIG. 5), according to some implementations, is illustrated. As shown, the hypnogram 600 includes a sleep-wake signal 601, a wakefulness stage axis 610, a REM stage axis 620, a light sleep stage axis 630, and a deep sleep stage axis 640. The intersection between the sleep-wake signal 601 and one of the axes 610-640 is indicative of the sleep stage at any given time during the sleep session.

The sleep-wake signal 601 can be generated based on physiological data associated with the user (e.g., generated by one or more of the sensors 210 described herein). The sleep-wake signal can be indicative of one or more sleep states, including wakefulness, relaxed wakefulness, microawakenings, a REM stage, a first non-REM stage, a second non-REM stage, a third non-REM stage, or any combination thereof. In some implementations, one or more of the first non-REM stage, the second non-REM stage, and the third non-REM stage can be grouped together and categorized as a light sleep stage or a deep sleep stage. For example, the light sleep stage can include the first non-REM stage and the deep sleep stage can include the second non-REM stage and the third non-REM stage. While the hypnogram 600 is shown in FIG. 6 as including the light sleep stage axis 630 and the deep sleep stage axis 640, in some implementations, the hypnogram 600 can include an axis for each of the first non-REM stage, the second non-REM stage, and the third non-REM stage. In other implementations, the sleep-wake signal can also be indicative of a respiration signal, a respiration rate, an inspiration amplitude, an expiration amplitude, an inspiration-expiration ratio, a number of events per hour, a pattern of events, or any combination thereof. Information describing the sleep-wake signal can be stored in the memory device 204.

The hypnogram 600 can be used to determine one or more sleep-related parameters, such as, for example, a sleep onset latency (SOL), wake-after-sleep onset (WASO), a sleep efficiency (SE), a sleep fragmentation index, sleep blocks, or any combination thereof.

The sleep onset latency (SOL) is defined as the time between the go-to-sleep time (t_{GTS}) and the initial sleep time (tₛₗₑₑₚ). In other words, the sleep onset latency is indicative of the time that it took the user to actually fall asleep after initially attempting to fall asleep. In some implementations, the sleep onset latency is defined as a persistent sleep onset latency (PSOL). The persistent sleep onset latency differs from the sleep onset latency in that the persistent sleep onset latency is defined as the duration time between the go-to-sleep time and a predetermined amount of sustained sleep. In some implementations, the predetermined amount of sustained sleep can include, for example, at least 10 minutes of sleep within the second non-REM stage, the third non-REM stage, and/or the REM stage with no more than 2 minutes of wakefulness, the first non-REM stage, and/or movement therebetween. In other words, the persistent sleep onset latency requires up to, for example, 8 minutes of sustained sleep within the second non-REM stage, the third non-REM stage, and/or the REM stage. In other implementations, the predetermined amount of sustained sleep can include at least 10 minutes of sleep within the first non-REM stage, the second non-REM stage, the third non-REM stage, and/or the REM stage subsequent to the initial sleep time. In such implementations, the predetermined amount of sustained sleep can exclude any micro-awakenings (e.g., a ten second micro-awakening does not restart the 10-minute period).

The wake-after-sleep onset (WASO) is associated with the total duration of time that the user is awake between the initial sleep time and the wake-up time. Thus, the wake-after-sleep onset includes short and micro-awakenings during the sleep session (e.g., the micro-awakenings MA₁ and MA₂ shown in FIG. 5), whether conscious or unconscious. In some implementations, the wake-after-sleep onset (WASO) is defined as a persistent wake-after-sleep onset (PWASO) that only includes the total durations of awakenings having a predetermined length (e.g., greater than 10 seconds, greater than 30 seconds, greater than 60 seconds, greater than about 5 minutes, greater than about 10 minutes, etc.)

The sleep efficiency (SE) is determined as a ratio of the total time in bed (TIB) and the total sleep time (TST). For example, if the total time in bed is 8 hours and the total sleep time is 7.5 hours, the sleep efficiency for that sleep session is 93.75%. The sleep efficiency is indicative of the sleep hygiene of the user. For example, if the user enters the bed and spends time engaged in other activities (e.g., watching TV) before sleep, the sleep efficiency will be reduced (e.g., the user is penalized). In some implementations, the sleep efficiency (SE) can be calculated based on the total time in bed (TIB) and the total time that the user is attempting to sleep. In such implementations, the total time that the user is attempting to sleep is defined as the duration between the go-to-sleep (GTS) time and the rising time described herein. For example, if the total sleep time is 8 hours (e.g., between 11 PM and 7 AM), the go-to-sleep time is 10:45 PM, and the rising time is 7:15 AM, in such implementations, the sleep efficiency parameter is calculated as about 94%.

The fragmentation index is determined based at least in part on the number of awakenings during the sleep session. For example, if the user had two micro-awakenings (e.g., micro-awakening MA₁ and micro-awakening MA₂ shown in FIG. 5), the fragmentation index can be expressed as 2. In some implementations, the fragmentation index is scaled between a predetermined range of integers (e.g., between 0 and 10).

The sleep blocks are associated with a transition between any stage of sleep (e.g., the first non-REM stage, the second non-REM stage, the third non-REM stage, and/or the REM) and the wakefulness stage. The sleep blocks can be calculated at a resolution of, for example, 30 seconds.

In some implementations, the systems and methods described herein can include generating or analyzing a hypnogram including a sleep-wake signal to determine or identify the enter bed time (t_{bed}), the go-to-sleep time (t_{GTS}), the initial sleep time (tₛₗₑₑₚ), one or more first micro-awakenings (e.g., MA₁ and MA₂), the wake-up time (t_{wake}), the rising time (tᵣᵢₛₑ), or any combination thereof based at least in part on the sleep-wake signal of a hypnogram.

In other implementations, one or more of the sensors 210 can be used to determine or identify the enter bed time (t_{bed}), the go-to-sleep time (tars), the initial sleep time (tₛₗₑₑₚ), one or more first micro-awakenings (e.g., MA₁ and MA₂), the wake-up time (t_{wake}), the rising time (tᵣᵢₛₑ), or any combination thereof, which in turn define the sleep session. For example, the enter bed time t_{bed} can be determined based on, for example, data generated by the motion sensor 218, the microphone 220, the camera 232, or any combination thereof. The go-to-sleep time can be determined based on, for example, data from the motion sensor 218 (e.g., data indicative of no movement by the user), data from the camera 232 (e.g., data indicative of no movement by the user and/or that the user has turned off the lights) data from the microphone 220 (e.g., data indicative of the using turning off a TV), data from the user device 260 (e.g., data indicative of the user no longer using the user device 260), data from the pressure sensor 212 and/or the flow rate sensor 214 (e.g., data indicative of the user turning on the respiratory therapy device 110, data indicative of the user donning the user interface 120, etc.), or any combination thereof.

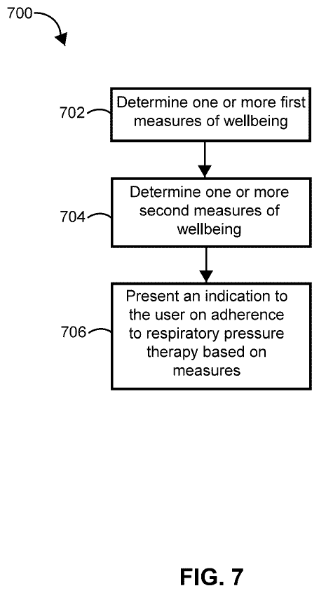

Referring to FIG. 7, a method 700 for determining and providing an indication of wellbeing of a user is illustrated, according to some implementations of the present disclosure. One or more steps of the method 700 can be implemented using any element or aspect of the system 100 (FIG. 1) described herein. For example, the control system 200 can implement the method 700.

At step 702, the method 700 includes determining one or more first measures of wellbeing of a user with sleep disordered breathing during an off period. The off period is when the user is not adhering to a respiratory pressure therapy. According to some implementations, the wellbeing of the user may relate more specifically to a fatigue level or an energy level of the user. Thus, the one or more first measures of wellbeing can relate to how much fatigue or energy the user has or is experiencing. However, more generally, the wellbeing of the user can relate to any broader quality of the user that is associated with the physical health, metal health, comfortability, etc. of the user, as further disclosed above.

The one or more first measures can be a single measure, which summarizes or generalizes the user's entire wellbeing. For example, and with respect to the wellbeing being more specifically associated with fatigue or energy, the one or more first measures can be a single measure that summarizes the user's fatigue or energy level on a scale of 1 to 10 or 1 to 100, with 10 or 100 representing less fatigue/more energy and 1 representing more fatigue/less energy.

Alternatively, the one or more first measures can be multiple, different measures relating to specific aspects of the user's wellbeing. For example, one first measure of wellbeing can relate to the user's mental fatigue/energy level. Another first measure of the wellbeing of the user can relate to the user's physical fatigue/energy level. Again, and as an example, the wellbeing of the user with respect to mental fatigue/energy can be a number from 1 to 10 or 1 to 100, and the wellbeing of the user with respect to physical fatigue/energy can be a number from 1 to 10 or 1 to 100. This implementation more specifically captures the ability for the user to feel different fatigue/energy levels for the mind and body, in contrast to a single first measure of wellbeing generalizing the wellbeing of the user with respect to metal and physical fatigue/energy with a single number.

The one or more first measures of wellbeing can be even more granular, with respect to physical (e.g., upper body/lower body, arms/abdomen/legs, etc.) and/or mental (e.g., critical thinking, mood, social, etc.) or can be related to other aspects of wellbeing, such as the health user, financial status of the user, etc.

The one or more first measures of wellbeing of the user are determined based on data associated with the user that is collected by any one or more of the sensors disclosed herein when the user is not adhering to a respiratory pressure therapy. There can be different levels of not adhering to the respiratory pressure therapy. According to some implementations, not adhering to the respiratory pressure therapy means that the user is not satisfying or exceeding every aspect of the respiratory pressure therapy. According to some implementations, not adhering to the respiratory pressure therapy means that the user is not satisfying or exceeding a majority of the aspects of the respiratory pressure therapy. According to some implementations, not adhering to the respiratory pressure therapy means that the user is satisfying (or not satisfying) a minimum of the aspects of the respiratory pressure therapy.

The adherence aspects of the respiratory pressure therapy can relate to the prescribed settings of the respiratory therapy system or device, such as the pressure, flow volume, humidity, temperature, etc. of the pressurized air being delivered by the respiratory therapy system or device. The adherence aspects of the respiratory pressure therapy can relate to the time on which the user is using the respiratory pressure therapy, such as an amount of time at night, a number of nights a week, and a number of nights/weeks a month, a number of consistent nights a month, etc. The goal of the one or more first measures is to quantify the wellbeing of the user when the user is not adhering to a respiratory pressure therapy for the purpose of, as described further below, comparing the wellbeing of the user when the user is adhering to a respiratory pressure therapy.

According to some implementations, the one or more first measures of wellbeing relate to sleep quality and are based on information obtained from one or more devices other than a sleep respiratory device providing the respiratory pressure therapy. For example, the one or more devices can be one or more health trackers, one or more smart devices, one or more home assistants, one or more exercise equipment, or a combination thereof. According to some aspects, the one or more devices belong to one or more health ecosystems.

At step 704, the method 700 includes determining one or more second measures of the wellbeing of the user during an on period. The on period is when the user is at least partially adhering to the respiratory pressure therapy. The one or more second measures of the wellbeing of the user can be the same type of measures as the one or more first measures. For example, the one or more second measures can be a single measure, if the one or more first measures are a single measure. Alternatively, the one or more second measures can be multiple different measures if the one or more first measures are multiple different measures. If the one or more first measures are with respect to physical and mental measures, the one or more second measures can be with respect to physical or mental measures.

According to some implementations, the period when the user is at least partially adhering to the respiratory pressure therapy can be the opposite of however the period was defined in step 702 above for when the user is not adhering to the respiratory pressure therapy. For example, the off period can be when the user is not using respiratory pressure therapy at all, and the on period can be when the user is using respiratory pressure therapy at all. Such a situation leaves no period that does not satisfy the off or on periods. Alternatively, the two periods can be more specifically defined. The off period can be when the user is not using any respiratory pressure therapy and the on period can be when the user is using respiratory pressure therapy exactly as described, such that periods in which the user is using respiratory pressure therapy not exactly as described are not considered off or on periods.

However, although generally described as an off period and an on period, the off and on periods can also be thought of as multiple different off periods and on periods that combined make up an off period and an on period, respectively. For example, over the period of a month, a user may adhere to respiratory pressure therapy for a first week, may not adhere to respiratory pressure therapy for a second week, may again adhere to respiratory pressure therapy for a third week, and then not adhere to respiratory pressure therapy for a fourth week. Although the first and third weeks and the second and fourth weeks are not continuous periods, together the first and third week can be considered an off period and the second and fourth week can be considered an on period.

According to some implementations, an on period can still be considered an on period despite the user not adhering to the respiratory pressure therapy during the on period. For example, if the user adheres to the respiratory pressure therapy for a continuous three months in a row and then has one night during which the user fails to use respiratory pressure therapy, followed by another three months in a row, that entire generally six month period can be considered an on period despite the one night off. The effect of a single night of not using respiratory pressure therapy within a period of six months may be negligible. Thus, an on period can include one or more periods where the user is not adhering to respiratory pressure therapy, such as, for example, 1%, or 2%, or 5%, or 10% of the time. Further, because respiratory pressure therapy typically occurs when the user is in bed and/or asleep, the determination as to whether the user is adhering or not adhering to the respiratory pressure therapy is with respect to only periods during a day that the user should be using respiratory pressure therapy.

According to some implementations, the one or more second measures of the wellbeing relate to sleep quality and are based on information obtained from sleep respiratory device providing the respiratory pressure therapy. The one or more second measures can be determined based on health information associated with the user and collected by a respiratory therapy system (e.g., respiratory therapy system 100) used by the user for at least partially adhering to the respiratory pressure therapy.

The one or more first measures of step 702, the one or more second measures of step 704, or a combination thereof can be determined based on one or more common objective measures of the wellbeing of the user, as described above. The one or more common objective measures can include, by way of example and without limitation, measures of consistency and/or punctuality in one or more daily routines, results of one or more cognitive tests, performance during one or more games, performance during one or more predefined routines, or a combination thereof. With respect to the one or more daily routines, such routines can include, for example, a daily routine of waking up and getting ready for work, including time to get out of bed, time to take a shower, and/or time to brush teeth. With respect to the one or more cognitive tests, such tests can include, for example, reaction time tests, reading time tests, and/or reading comprehension tests.

Alternatively, or in combination with the objective measures, the one or more first measures, the one or more second measures, or a combination thereof can be determined based on one or more subjective measures provided by the user. Such subject measures can relate to health information associated with the user and be collected by one or more user devices (e.g., user devices 260). The health information that is subjective can be the user subjectively describing how the user feels with respect to a period of time, such as the present, the past 24 hours, the past week, etc. Alternatively, or in addition, the objective measures also can relate to health information associated with the user and collected by one or more user devices.

At step 706, the method 700 includes presenting an indication to the user on how at least partially adhering to the respiratory pressure therapy improves the wellbeing of the user by comparing the one or more first measures to the one or more second measures. The indication reveals to the user how adhering to the respiratory pressure therapy has improved the user's wellbeing.

According to some implementations, the indication can provide a direct one-to-one comparison of the first and second measures, such as being a difference between the one or more first measures and the one or more second measures. The indication can alternatively provide a summary of a one-to-one comparison of the first and second measures.

According to some implementations, the indication can be presented on a respiratory therapy device (e.g., respiratory therapy device 110) the user uses for the respiratory pressure therapy. According to some implementations, the indication is presented across multiple devices associated with the user. When presented across multiple devices, the format of the indication can vary depending on a device type for the multiple devices associated with the user. For example, the indication can present more information when presented on a display of a desktop or laptop or tablet as compared to a smartphone. According to some implementations, a format of the indication can be based on a health history of the user, one or more current health issues of the user, a type of sleep disordered breathing, or a combination thereof.

According to some implementations, each type of the one or more first measures can be normalized among its respective type of the first measures to compare the respective type of the first measure with the one or more first measures. Similarly, each type of the one or more second measures can be normalized among its respective type of the second measures to compare the respective type of the second measure with the one or more second measures. Further, each type of first measures can be normalized among its respective type of the first measures to compare the respective type of the first measures with the one or more second measures. Each type of the second measures is normalized among its respective type of the second measure to compare the respective type of the second measure with the one or more first measures. The normalization of the first and second measures allows for a comparison the measures despite the measures being different metric modalities. For example, the normalization allows for the comparison of time-based objective measures to feeling-based subject measures.

Before or after step 706, according to some implementations, one or more contributing factors that affect the wellbeing of the user during the off period, the on period, or a combination thereof can be determined. The one or more contributing factors can include, for example, one or more environmental factors. The one or more contributing factors can be determined by any of the above-described devices or systems that collect other information related to the wellbeing of the user. Once the contributing factors are determined, the indication to the user can correlate the one or more contributing factors with the one or more first measures of wellbeing, the one or more second measures of wellbeing, or a combination thereof. This provides information to the user on how the contributing factors affect the wellbeing.

### Alternative Implementation Section

Implementation 1. A method comprising determining one or more first measures of wellbeing of a user with sleep disordered breathing during an off period when the user is not adhering to a respiratory pressure therapy; determining one or more second measures of the wellbeing of the user during an on period when the user is at least partially adhering to the respiratory pressure therapy; and presenting an indication to the user on how at least partially adhering to the respiratory pressure therapy improves the wellbeing of the user by comparing the one or more first measures to the one or more second measures.

Implementation 2. The method of implementation 1, wherein the one or more first measures of wellbeing relate to sleep quality and are based on information obtained from one or more devices other than a sleep respiratory device providing the respiratory pressure therapy, and the one or more second measures of the wellbeing relate to sleep quality and are based on information obtained from sleep respiratory device providing the respiratory pressure therapy.

Implementation 3. The method of implementation 1, wherein each type of first measure of the one or more first measures is normalized among its respective type of the first measure to compare the respective type of the first measure with the one or more first measures

Implementation 4. The method of implementation 3, wherein each type of second measure of the one or more second measures is normalized among its respective type of the second measure to compare the respective type of the second measure with the one or more second measures.

Implementation 5. The method of implementation 4, wherein each type of first measure of the one or more first measures is normalized among its respective type of the first measure to compare the respective type of the first measure with the one or more second measures, and each type of second measure of the one or more second measures is normalized among its respective type of the second measure to compare the respective type of the second measure with the one or more first measures.

Implementation 6. The method of implementation 1, wherein a format of the indication is based on a health history of the user, one or more current health issues of the user, a type of sleep disordered breathing, or a combination thereof.

Implementation 7. The method of implementation 1, wherein the indication is presented on a respiratory therapy device the user uses for the respiratory pressure therapy.

Implementation 8. The method of implementation 1, wherein the indication is presented across multiple devices associated with the user.

Implementation 9. The method of implementation 8, wherein a format of the indication varies depending on a device type for the multiple devices associated with the user.

Implementation 10. The method of implementation 1, further comprising: collecting information from one or more devices associated with the user separate from a respiratory therapy device that provides the positive airway pressure to the user.

Implementation 11. The method of implementation 10, wherein the information is collected at least during the off period.

Implementation 12. The method of implementation 11, wherein the information is collected during the off period and the on period.

Implementation 13. The method of implementation 9, wherein the one or more devices are one or more health trackers, one or more smart devices, one or more home assistants, one or more exercise equipment, or a combination thereof.

Implementation 14. The method of implementation 9, wherein the one or more devices belong to one or more health ecosystems.

Implementation 15. The method of implementation 1, wherein the one or more first measures, the one or more second measures, or a combination thereof are determined based on one or more common objective measures of the wellbeing of the user.

Implementation 16. The method of implementation 15, wherein the one or more common objective measures include measures of consistency and/or punctuality in one or more daily routines, results of one or more cognitive tests, performance during one or more games, performance during one or more predefined routines, or a combination thereof.

Implementation 17. The method of implementation 16, wherein the one or more daily routines include a daily routine of waking up and getting ready for work, including time to get out of bed, time to take a shower, and time to brush teeth.

Implementation 18. The method of implementation 17, wherein the one or more cognitive tests include reaction time tests, reading time tests, and reading comprehension tests.

Implementation 19. The method of implementation 1, wherein the one or more first measures, the one or more second measures, or a combination thereof are determined based on one or more subjective measures provided by the user.

Implementation 20. The method of implementation 1, wherein the one or more first measures, the one or more second measures, or a combination thereof are determined based on health information associated with the user and collected by one or more smart devices.

Implementation 21. The method of implementation 20, wherein the one or more second measures are determined based on health information associated with the user and collected by a respiratory therapy system used by the user for at least partially adhering to the respiratory pressure therapy.

Implementation 22. The method of implementation 21, wherein the one or more first measures of wellbeing and the one or more second measures of wellbeing relate to fatigue of the user.

Implementation 23. The method of implementation 1, further comprising: determining one or more contributing factors that affect the wellbeing of the user during the off period, the on period, or a combination thereof, wherein the indication to the user correlates the one or more contributing factors with the one or more first measures of wellbeing, the one or more second measures of wellbeing, or a combination thereof.

Implementation 24. The method of implementation 23, wherein the one or more contributing factors include one or more environmental factors.

Implementation 25. A system comprising: a control system comprising one or more processors; and a memory having stored thereon machine readable instructions; wherein the control system is coupled to the memory, and the method of any one of claims 1 to ## is implemented when the machine executable instructions in the memory are executed by at least one of the one or more processors of the control system.

Implementation 26. A system for communicating one or more indications to a user, the system comprising a control system configured to implement the method of any one of implementations 1 to 25.

Implementation 27. A computer program product comprising instructions which, when executed by a computer, cause the computer to carry out the method of any one of implementations 1 to 25.

Implementations 28. The computer program product of implementation 27, wherein the computer program product is a non-transitory computer readable medium.

Implementation 29. A system comprising: a memory storing machine-readable instructions; and a control system including one or more processors configured to execute the machine-readable instructions to perform the steps of: determining one or more first measures of wellbeing of a user with sleep disordered breathing during an off period when the user is not adhering to a respiratory pressure therapy; determining one or more second measures of the wellbeing of the user during an on period when the user is at least partially adhering to the respiratory pressure therapy; and presenting an indication to the user on how at least partially adhering to the respiratory pressure therapy improves the wellbeing of the user by comparing the one or more first measures to the one or more second measures.

Implementation 30. The system of implementation 29, wherein the one or more first measures of wellbeing relate to sleep quality and are based on information obtained from one or more devices other than a sleep respiratory device providing the respiratory pressure therapy, and the one or more second measures of the wellbeing relate to sleep quality and are based on information obtained from sleep respiratory device providing the respiratory pressure therapy.

Implementation 31. The system of implementation 29, wherein each type of first measure of the one or more first measures is normalized among its respective type of the first measure to compare the respective type of the first measure with the one or more first measures

Implementation 32. The system of implementation 31, wherein each type of second measure of the one or more second measures is normalized among its respective type of the second measure to compare the respective type of the second measure with the one or more second measures.

Implementation 33. The system of implementation 32, wherein each type of first measure of the one or more first measures is normalized among its respective type of the first measure to compare the respective type of the first measure with the one or more second measures, and each type of second measure of the one or more second measures is normalized among its respective type of the second measure to compare the respective type of the second measure with the one or more first measures.

Implementation 34. The system of implementation 29, wherein a format of the indication is based on a health history of the user, one or more current health issues of the user, a type of sleep disordered breathing, or a combination thereof.

Implementation 35. The system of implementation 29, wherein the indication is presented on a respiratory therapy device the user uses for the respiratory pressure therapy.

Implementation 36. The system of implementation 29, wherein the indication is presented across multiple devices associated with the user.

Implementation 37. The system of implementation 36, wherein a format of the indication varies depending on a device type for the multiple devices associated with the user.

Implementation 38. The system of implementation 29, wherein the one or more processors are further configured to execute the machine-readable instructions to perform the step of: collecting information from one or more devices associated with the user separate from a respiratory therapy device that provides the positive airway pressure to the user.

Implementation 39. The system of implementation 38, wherein the information is collected at least during the off period.

Implementation 40. The system of implementation 39, wherein the information is collected during the off period and the on period.

Implementation 41. The system of implementation 38, wherein the one or more devices are one or more health trackers, one or more smart devices, one or more home assistants, one or more exercise equipment, or a combination thereof.

Implementation 42. The system of implementation 38, wherein the one or more devices belong to one or more health ecosystems.

Implementation 43. The system of implementation 29, wherein the one or more first measures, the one or more second measures, or a combination thereof are determined based on one or more common objective measures of the wellbeing of the user.

Implementation 44. The system of implementation 43, wherein the one or more common objective measures include measures of consistency and/or punctuality in one or more daily routines, results of one or more cognitive tests, performance during one or more games, performance during one or more predefined routines, or a combination thereof.

Implementation 45. The system of implementation 44, wherein the one or more daily routines include a daily routine of waking up and getting ready for work, including time to get out of bed, time to take a shower, and time to brush teeth.

Implementation 46. The system of implementation 45, wherein the one or more cognitive tests include reaction time tests, reading time tests, and reading comprehension tests.

Implementation 47. The system of implementation 29, wherein the one or more first measures, the one or more second measures, or a combination thereof are determined based on one or more subjective measures provided by the user.

Implementation 48. The system of implementation 29, wherein the one or more first measures, the one or more second measures, or a combination thereof are determined based on health information associated with the user and collected by one or more smart devices.

Implementation 49. The system of implementation 48, wherein the one or more second measures are determined based on health information associated with the user and collected by a respiratory therapy system used by the user for at least partially adhering to the respiratory pressure therapy.

Implementation 50. The system of implementation 29, wherein the one or more first measures of wellbeing and the one or more second measures of wellbeing relate to fatigue of the user.

Implementation 51. The system of implementation 29, wherein the one or more processors are further configured to execute the machine-readable instructions to perform the steps of: determining one or more contributing factors that affect the wellbeing of the user during the off period, the on period, or a combination thereof, wherein the indication to the user correlates the one or more contributing factors with the one or more first measures of wellbeing, the one or more second measures of wellbeing, or a combination thereof.

Implementation 52. The system of implementation 51, wherein the one or more contributing factors include one or more environmental factors.

One or more elements or aspects or steps, or any portion(s) thereof, from one or more of any of the above implementations above can be combined with one or more elements or aspects or steps, or any portion(s) thereof, from one or more of any of the other above implementations or combinations thereof, to form one or more additional implementations and/or claims of the present disclosure.

While the present disclosure has been described with reference to one or more particular embodiments or implementations, those skilled in the art will recognize that many changes may be made thereto without departing from the spirit and scope of the present disclosure. Each of these implementations and obvious variations thereof is contemplated as falling within the spirit and scope of the present disclosure. It is also contemplated that additional implementations according to aspects of the present disclosure may combine any number of features from any of the implementations described herein.

What is claimed is:

1. A method comprising:
determining one or more first measures of wellbeing of a user with sleep disordered breathing during an off period when the user is not adhering to a respiratory pressure therapy;
determining one or more second measures of the wellbeing of the user during an on period when the user is at least partially adhering to the respiratory pressure therapy; and
presenting an indication to the user on how at least partially adhering to the respiratory pressure therapy improves the wellbeing of the user by comparing the one or more first measures to the one or more second measures.

2. The method of claim 1, wherein the one or more first measures of wellbeing relate to sleep quality and are based on information obtained from one or more devices other than a sleep respiratory device providing the respiratory pressure therapy, and the one or more second measures of the wellbeing relate to sleep quality and are based on information obtained from sleep respiratory device providing the respiratory pressure therapy.

3. The method of claim 1, wherein each type of first measure of the one or more first measures is normalized among its respective type of the first measure to compare the respective type of the first measure with the one or more first measures.

4. The method of claim 3, wherein each type of second measure of the one or more second measures is normalized among its respective type of the second measure to compare the respective type of the second measure with the one or more second measures.

5. The method of claim 4, wherein each type of first measure of the one or more first measures is normalized among its respective type of the first measure to compare the respective type of the first measure with the one or more second measures, and each type of second measure of the one or more second measures is normalized among its respective type of the second measure to compare the respective type of the second measure with the one or more first measures.

6. The method of claim 1, wherein a format of the indication is based on a health history of the user, one or more current health issues of the user, a type of sleep disordered breathing, or a combination thereof.

7. The method of claim 1, wherein the indication is presented on a respiratory therapy device the user uses for the respiratory pressure therapy or across multiple devices associated with the user.

8. The method of claim 1, further comprising collecting information from one or more devices associated with the user separate from a respiratory therapy device that provides the positive airway pressure to the user.

9. The method of claim 8, wherein the information is collected at least during the off period.

10. The method of claim 1, wherein the one or more first measures, the one or more second measures, or a combination thereof are determined based on one or more common objective measures of the wellbeing of the user, wherein the one or more common objective measures include measures of consistency and/or punctuality in one or more daily routines, results of one or more cognitive tests, performance during one or more games, performance during one or more predefined routines, or a combination thereof.

11. The method of claim 10, wherein the one or more daily routines include a daily routine of waking up and getting ready for work, including time to get out of bed, time to take a shower, and time to brush teeth.

12. The method of claim 10, wherein the one or more cognitive tests include reaction time tests, reading time tests, and reading comprehension tests.

13. The method of claim 1, wherein the one or more first measures, the one or more second measures, or a combination thereof are determined based on one or more subjective measures provided by the user.

14. The method of claim 1, wherein the one or more first measures, the one or more second measures, or a combination thereof are determined based on health information associated with the user and collected by one or more smart devices.

15. The method of claim 14, wherein the one or more second measures are determined based on health information associated with the user and collected by a respiratory therapy system used by the user for at least partially adhering to the respiratory pressure therapy.

16. The method of claim 1, wherein the one or more first measures of wellbeing and the one or more second measures of wellbeing relate to fatigue of the user.

17. The method of claim 1, further comprising:
determining one or more contributing factors that affect the wellbeing of the user during the off period, the on period, or a combination thereof,
wherein the indication to the user correlates the one or more contributing factors with the one or more first measures of wellbeing, the one or more second measures of wellbeing, or a combination thereof.

18. The method of claim 17, wherein the one or more contributing factors include one or more environmental factors.

19. A non-transitory computer readable medium computer program product comprising instructions which, when executed by a computer, cause the computer to carry out:
determining one or more first measures of wellbeing of a user with sleep disordered breathing during an off period when the user is not adhering to a respiratory pressure therapy;
determining one or more second measures of the wellbeing of the user during an on period when the user is at least partially adhering to the respiratory pressure therapy; and
presenting an indication to the user on how at least partially adhering to the respiratory pressure therapy improves the wellbeing of the user by comparing the one or more first measures to the one or more second measures.

20. A system comprising:
a memory that stores machine-readable instructions; and
a control system that includes one or more processors, the one or more processors are configured to execute the machine-readable instructions to:
determine one or more first measures of wellbeing of a user with sleep disordered breathing during an off period when the user is not adhering to a respiratory pressure therapy;
determine one or more second measures of the wellbeing of the user during an on period when the user is at least partially adhering to the respiratory pressure therapy; and
present an indication to the user on how at least partially adhering to the respiratory pressure therapy improves the wellbeing of the user by comparing the one or more first measures to the one or more second measures.
